(19) United States
(12) Patent Application Publication
(10) Pub. No.: US 2012/0100171 A1
(43) Pub. Date: Apr. 26, 2012

(54) HAPTEN-CARRIER CONJUGATES WITH BACTERIAL TOXINS HAVING A SIGNAL PEPTIDE AS CARRIER AND THEIR USE IN IMMUNOGENIC COMPOSITIONS

(76) Inventor: William Henry, County Bucks (GB)

(21) Appl. No.: 13/255,262

(22) PCT Filed: Mar. 9, 2010
(86) PCT No.: PCT/EP2010/052997

§ 371 Date: Dec. 19, 2011

Related U.S. Application Data
(60) Provisional application No. 61/209,629, filed on Mar. 9, 2009.

(51) Int. Cl. A61K 39/385; A61P 37/04; A61P 25/30; C07K 14/28

(52) U.S. Cl. 424/190.1; 530/403; 530/405

(57) ABSTRACT

This invention relates to immunogenic compositions for inducing an immune response against an antigen of interest. In particular, the invention provides immunogenic compositions comprising an antigen-carrier conjugate, wherein the carrier is a bacterial toxin that contains a signal peptide. The invention also provides methods of generating immunogenic compositions with enhanced immunogenicity, comprising adding a signal peptide to the bacterial carrier in an antigen-carrier conjugate, such as a hapten-carrier conjugate. The invention also provides methods for inducing an immune response to a hapten in a subject using these immunogenic compositions.

This application is a national stage entry under 35 U.S.C. §371 of International Application No. PCT/EP2010/052997, filed Mar. 9, 2010, which itself claims the benefit of U.S. Provisional Patent Application No. 61/209,629, filed Mar. 9, 2009, the contents of each of which is incorporated herein by reference in its entirety.

### 1. FIELD OF THE INVENTION

This invention relates to immunogenic compositions for inducing an immune response against an antigen of interest. In particular, the invention provides immunogenic compositions comprising an antigen-carrier conjugate, wherein the carrier is a bacterial toxin that contains a signal peptide. The invention also provides methods of generating immunogenic compositions with enhanced immunogenicity, comprising adding a signal peptide to the bacterial carrier in an antigen-carrier conjugate, such as a hapten-carrier conjugate. The invention also provides methods for inducing an immune response to a hapten in a subject using these immunogenic compositions.

### 2. BACKGROUND OF THE INVENTION

Bacterial toxin-hapten conjugates are currently in clinical trials as therapeutics for drug addiction. In these ongoing trials, when an immunogenic composition containing the hapten/drug, e.g., a cocaine-bacterial toxin conjugate or a nicotine-bacterial toxin conjugate, is administered to the addicted individual, antibodies specific to the drug are elicited. For example, when the therapeutic composition is a cocaine-carrier conjugate, treatment induces an anti-cocaine antibody response which reduces cocaine in the bloodstream or mucosal tissue of a subject, thereby reducing the psychologically addictive properties of the drug. Treatment with nicotine-carrier conjugates induces anti-nicotine antibodies and diminishes the gratification from the use of nicotine.

### 3. SUMMARY OF THE INVENTION

The initial clinical success of drug-carrier conjugates in which the carrier is a bacterial toxin has prompted a need for drug-bacterial toxin conjugates with even greater immunogenicity. Generation of drug-bacterial toxin conjugates with enhanced immunogenicity have the clinical advantage of being administered in fewer doses, at lower doses, and/or with a greater interval of time between doses. The availability of conjugates with greater potency will also greatly facilitate the large-scale production of such vaccines. The invention is based in part on the discovery that hapten-carrier conjugates comprising nicotine conjugated to a cholera toxin B (CTB) carrier that contains a signal peptide have enhanced immunogenicity compared to nicotine-CTB conjugates that lack the signal peptide.

Provided herein are immunogenic compositions comprising a bacterial toxin for which immunogenicity is enhanced by containing a signal peptide. In one embodiment, the signal peptide is the bacterial toxin's endogenous signal peptide or a fragment thereof. In one embodiment, the signal peptide comprises the amino acid sequence Ala-Pro-Gly-Tyr-Ala-His-Gly (SEQ ID NO: 1). In other embodiments, the signal peptide comprises the amino acid sequence Gly-Tyr-Ala-His-Gly (SEQ ID NO: 2). In other embodiments, the signal peptide comprises the amino acid sequence Tyr-Ala-His-Gly (SEQ ID NO: 3). In other embodiments, the signal peptide comprises the amino acid sequence Ala-His-Gly. In other embodiments, the signal peptide comprises the amino acid sequence His-Gly. In other embodiments, the signal peptide comprises a single Gly residue. In some embodiments of the invention, the immunogenic composition comprises a mixture of one or more of these peptides.

In another embodiment of the invention, the immunogenic composition comprises a signal peptide-containing bacterial toxin selected from the group comprising a bacterial ADP-ribosylating exotoxin subunit peptide, cholera toxin B (CTB) Escherichia coli heat-labile enterotoxin (LT), diphtheria toxin, tetanus toxoid, pertussis toxin and filamentous hemagglutinin, shiga toxin, and pseudomonas exotoxin. Other useful bacterial toxin carriers include any bacterial toxin with the ability to enhance a mucosal response, for example, any toxin in the LTB family of bacterial toxins.

In some embodiments, the bacterial toxin carrier contains its endogenous signal peptide. In some embodiments, the bacterial toxin carrier of the invention, such as CTB or any of the aforementioned bacterial toxins, contains a heterologous signal peptide.

This invention provides immunogenic compositions comprising an antigen and a carrier, wherein the carrier is a signal peptide-containing bacterial toxin and the antigen is any molecule against which it is desired to produce an immune response. In an embodiment of the invention, the antigen is conjugated to the carrier. In some embodiments, the antigen is a hapten. This invention is based in part on the discovery that signal peptide-containing bacterial toxin carriers impart a greater immune response against conjugated haptens than bacterial toxin carriers that lack a signal peptide. In some embodiments, the hapten is a hapten known to be immunogenic when present in a conjugate with a bacterial toxin that lacks a signal peptide or for which the signal peptide is unstable, and for which enhanced immunity is desired.

In some embodiments of the invention, the hapten conjugated to the bacterial toxin that contains a signal peptide is nicotine or a derivative of nicotine. In some embodiments, the hapten is cocaine or another drug of addition. In some embodiments, the hapten is an antigen of a pathogen. In some embodiments, the antigen in the antigen-bacterial toxin conjugate is not a hapten.

This invention also provides methods for inducing immunity to a hapten in a subject using the aforementioned immunogenic compositions.

This invention also provides methods of generating immunogenic compositions comprising bacterial toxin carriers containing a signal peptide. In some embodiments, the method involves expressing the bacterial toxin in cells in which the bacterial toxin contains a signal peptide. In some embodiments, the bacterial toxin is CTB and the cells in which it is produced is the Vibrio cholerae 213 strain.

### 4. BRIEF DESCRIPTION OF THE DRAWINGS

FIG. 1. Amino acid sequence of rCTB (recombinant cholera toxin B) (SEQ ID NO: 4).

FIG. 2. Comparison of the two rCTB expression systems in V. cholerae 213 and 401. When produced in the 213 strain, forms of rCTB are detected that contain a signal sequence of up to 7 amino acids in length (SEQ ID NOS: 5-10). No stable signal sequence is detected when rCTB is produced in the 401 strain (SEQ ID NO: 11).

FIG. 3. (A) Representation of a number of possible, arbitrarily labeled, “branches” of a hapten-carrier conjugate identified for ease of understanding suitable compounds and conjugates for use in the practice of the instant invention. (B) Representation of possible, arbitrarily labeled, “branches” of a hapten-carrier conjugate identified for ease of understanding suitable compounds and conjugates used in the practice of the instant inventions, wherein Q′ is a modified T-cell epitope-containing carrier, such as a modified protein carrier.

FIG. 4. Representation of nicotine and some of its derivatives and metabolites useful in preparation of the immunogenic compositions of the invention.

### 5. DETAILED DESCRIPTION OF THE INVENTION

Using the methods and compositions of the present invention, and more particularly, the techniques set out herein, one skilled in the art can link any selected signal peptide-containing bacterial toxin carrier with any selected antigen, for example, a hapten, to make an antigen-carrier conjugate of the instant invention. Any number of carriers and antigens and/or haptens may be present in a single conjugate molecule of the invention.

5.1 Carriers for Use in the Immunogenic Compositions of the Invention

This invention provides immunogenic compositions comprising antigen-carrier conjugates, such as hapten-carrier conjugates, in which the carrier is a bacterial toxin that contains a signal peptide. In some embodiments, the bacterial toxin contains its endogenous signal peptide. In some embodiments, the bacterial toxin has been engineered to contain a signal peptide. In some embodiments, the bacterial toxin retains its signal peptide, e.g., it is not removed from the bacterial toxin.

In the present invention, the immunogenic composition comprises an antigen, such as a hapten, and a carrier, in which the carrier is a signal peptide-containing bacterial toxin. In an embodiment of the invention, the antigen is conjugated to the carrier. In some embodiments, the bacterial toxin is from a gram-negative bacteria. In some embodiments, the toxin is from a gram-positive bacteria. In some embodiments, the gram-negative bacteria is Escherichia coli. In some embodiments, the bacteria is the gram-positive bacillus, staphylococcus, streptococcus, streptomyces, or mollicutes (mycoplasma). In some embodiments, the carrier is a bacterial exotoxin. In some embodiments, the carrier is a bacterial toxin that has been modified to reduce its toxicity. Bacterial toxins for use in accordance with the invention include, but are not limited to, cholera toxin, preferably CTB (including recombinant CTB (rCTB)), Escherichia coli toxins such as heat-labile enterotoxin (LT), heat-stable exotoxin (ST), cytotoxic necrotizing factor (CNF), cytolethal distending toxin (CLDT), or enteroaggregative E. coli heat-stable toxin (EAST), diphtheria toxin (Dtx), tetanus toxin, shiga toxins, botulinum toxin, staphylococci toxins, such as Staphylococcus aureus alpha toxin, Exfoliatin B or leukocidin, staphylococcal toxic shock syndrome toxin (TSST-1), staphylococcus enterotoxins or exfoliative toxins, streptococci toxins, such as pneumolysin of Streptococcus pneumoniae, streptolysin O, Erythrogenic toxin (streptococcal pyrogenic exotoxin (SPE)), and other pyrogenic toxins of Streptococcus pyogenes, clostridial toxins, such as toxin A/toxin B of Clostridium difficile, Iota family, C2 family (toxins C and D), or C3 toxins, or neurotoxins A-G of Clostridium botulinum, alpha toxin, beta-2 toxin, or perfringiolysin O of Clostridium perfringens (Perfringens enterotoxin), enterotoxin of Bacteroides fragilis, Aeromonas hydrophila/aerolysin, filamentous hemagglutinin (FHA) of Bordatella pertussis, Clostridium or Bacillus binary toxins, streptokinase, the adenylate cyclase toxin of Bordatella pertussis (pertussis AC; “pertussis toxin”) or its dermonecrotic toxin, Bacillus anthracis edema factor (EF), anthrax toxin (lethal factor (LF)), hemolysin of Escherichia coli, listeriolysin of Listeria monocytogenes, and Pseudomonas exotoxin (exotoxin A). In some embodiments, a bacterial ADP-ribosylating exotoxin is preferably used, such as, for example, cholera toxin, diphtheria toxin, pertussis toxin, Pseudomonas exotoxin A, or E. coli LT. In some embodiments, the catalytic subunit (usually, the “A” subunit) of the bacterial ADP-ribosylating exotoxin is used as carrier. In other embodiments, the receptor-binding subunit of the bacterial ADP-ribosylating exotoxin (usually, the “B” subunit) is preferred. In yet other embodiments, both subunits or a fragment or fragments thereof are used as a carrier. In some embodiments, a bacterial pore-forming toxin is used, such as perfringiolysin O, hemolysin, listeriolysin, anthrax EF, alpha toxin, pneumolysin, streptolysin O, or leukocidin. In some embodiments, the carrier is a pyrogenic exotoxin, or a modified form thereof, such as staphylococcal enterotoxins serotypes A-E, G, and H, group A streptococcal pyrogenic exotoxins A-C, staphylococcal exfoliatin toxin, and staphylococcal TSST-1. Particularly useful bacterial toxin carriers include any bacterial toxin with the ability to enhance a mucosal immune response, for example, CTB or any toxin in the E. coli heat-labile enterotoxin (LTB) family of bacterial toxins.

In some embodiments of the invention, the bacterial toxin carrier contains its endogenous signal peptide, i.e., the signal peptide present on the bacterial toxin when the protein is translated, or a fragment thereof. In other embodiments, the bacterial toxin carrier contains a signal peptide present in the same bacteria from which the toxin is derived, but the signal peptide is normally present on a different protein in that bacteria. In some embodiments, a toxin carrier or immunogenic fragment thereof is used which does not normally have a signal peptide and to which a signal peptide is added. In one such embodiment, tetanus toxin is engineered to contain a signal peptide.

In some embodiments, the bacterial toxin carrier of the invention, such as CTB or any of the aforementioned bacterial toxins, contains a heterologous signal peptide. In some embodiments, the heterologous signal peptide is a secretion leader sequence such as those known to one of skill in the art and include, for example, the tissue plasminogen activator (tpa) leader sequence, tobacco pathogenesis-related 1b (PR1b) signal peptide, or any another signal peptide known in the art or fragment thereof. In some embodiments, the signal peptide is a bacterial signal peptide. Bacterial signal peptides for use in the practice of the invention can be selected from the group comprising CTB signal peptide (the fragments described herein or, e.g., Accession No. P01556), E. coli heat labile enterotoxin subunit B signal peptide (e.g., Accession No. P13811), diphtheria toxin signal peptide (e.g., Accession No. P00588), pertussis toxin signal peptides (see, for example, U.S. Pat. No. 4,883,761 or Accession Nos. PO4977 and PO4978), Shiga toxin signal peptides (see, e.g., Accession Nos. Q9FBI2 and Q7BQ98), Pseudomonas exotoxin A (e.g., Accession No. P11439), the long signal peptide of FHA toxin, the TSST-1 signal peptide, Streptococcal a toxin signal peptide, Staphylococcal protein A signal peptide, Clostridium perfringens alpha toxin signal peptide, Clostridium perfringens beta-2 toxin signal peptide (see, for example, U.S. Pat. No. 7,144,998), signal peptides of the A and B subunits of Clostridium and Bacillus binary toxins, among others. See Table 1 below for a non-limiting list of exemplary bacterial toxin signal peptides for use in the invention. The full-length signal peptide sequence is shown; however, fragments, for example, C-terminal fragments, of 10 amino acids or less, or 5 amino acids or less, etc., may be used in the practice of the invention. The signal peptide or fragment thereof may be 1-3 amino acids in length, 3-5 amino acids in length, 5-10, preferably 7, amino acids in length, 10 to 15 amino acids in length, 15 to 20 amino acids in length, 20 to 25 amino acids in length, 25 to 30 amino acids in length, 30 to 35 amino acids in length, 35 to 40 amino acids in length, or 40 to 50, or more amino acids in length. In other embodiments, the signal peptide or fragment thereof as used in the invention has 90% or better, 85% or better, 80% or better, 75% or better, 70% or better, 65% or better, or 60% or better sequence identity with the naturally occurring signal peptide.

In some embodiments, a CTB signal peptide or fragment thereof, such as described in Section 5.1.1 below, is the heterologous signal peptide on a bacterial toxin carrier other than CTB.

For the purposes of this application, a signal peptide is a short (usually 3-60 amino acids long, but can be shorter if a fragment is used, and can be longer, e.g., as in FHA) peptide chain that directs the transport, post-translationally or co-translationally, of a protein. Signal peptides may also be known as targeting signals, signal sequences, transit peptides, or localization signals. Generally, the amino acid sequences of signal peptides direct proteins to the endoplasmic reticulum in eukaryotes and to the cell membrane in prokaryotes. For example, many bacterial exotoxins are synthesized with an amino terminal signal peptide consisting of a few (1-3) charged amino acids and a stretch of (14-20) hydrophobic amino acids. The signal peptide may bind and insert into the cytoplasmic membrane during translation so that the toxin is secreted during its synthesis. Usually, the signal peptide is cleaved as the toxin is released into the periplasm. Alternatively, the toxin may be synthesized intracytoplasmically, then bound to a leader sequence for passage across the membrane. Signal peptides that function in such manners, or those that function through other mechanisms, are contemplated for use in the invention.

TABLE 1
Exemplary bacterial toxin signal peptides for use in the invention

| Bacterial | Accession | Signal peptide amino acid sequence |
|---|---|---|
| toxin protein | No. | (amino acid position in protein) |
| CTB | P01556 | MIKLKFGVFF TVLLSSAYAH G (1-21) (SEQ ID NO: 12) |
| E. coli LT-B | P13811 | MNKVKFYVLF TALLSSLCAH G (1-21) (SEQ ID NO: 13) |
| Diphtheria | P00588 | MLVRGYVVSR KLFASILIGA LLGIGAPPSA HA (1-32) (SEQ ID NO: 14) |
| toxin | | |
| Pertussis | P04977 | MRCTRAIRQT ARTGWLTWLA ILAVTAPVTS PAWA (1-34) (SEQ ID NO: 15) |
| toxin-A | | |
| Pertussis | P04978 | MPIDRKTLCH LLSVLPLALL GSHVARA (1-27) (SEQ ID NO: 16) |
| toxin-B | | |
| Shiga toxin-A | Q9FBI2 | MKIIIFRVLT FFFVIFSVNV VA (1-22) (SEQ ID NO: 17) |
| Shiga toxin-B | Q7BQ98 | MKKTLLIAAS LSFFSASALA (1-20) (SEQ ID NO: 18) |
| Pseudomonas | P11439 | MHLTPHWIPL VASLGLLAGG SFASA (1-25) (SEQ ID NO: 19) |
| exotoxin A | | |

Any bacterial toxin carrier known in the art can be modified to contain a signal peptide for use in accordance with the present invention. Standard techniques for adding a signal peptide sequence to a bacterial toxin include recombinant DNA techniques or any other technique that allows engineering of proteins at either the DNA, RNA, or protein level. In order to ensure enhanced stability of a signal peptide, whether the signal peptide is endogenous to the carrier or added by recombinant DNA technology, one of skill in the art can produce the bacterial toxin carrier of choice in alternative systems, such as mammalian cells, insect cells (using, for example, a baculovirus expression system), bacterial cells, preferably, for a CTB signal peptide, Vibrio cholerae strain 213, or plant cells, for example, by transgenic expression in tobacco plants, and choose the optimal system from which to isolate the signal peptide-bearing bacterial toxin. Other methods for enhancing production and isolation of signal peptide-bearing bacterial toxins include expression in systems in which protease inhibitors, for example, signal peptide peptidase inhibitors, are added either during and/or after protein expression or expression in systems in which the apparatus for translocation or post-translocation processing is defective. Any other system optimized so that signal peptide-bearing bacterial toxins accumulate and/or have enhanced stability may be used in accordance with the invention.

In some embodiments, the bacterial toxin carrier of the instant invention contains at least one T cell epitope which is capable of stimulating the T cells of the subject, which in turn help the B cells initiate and maintain sustained antibody production to portions of the entire conjugate, including the hapten portion. Thus, since a carrier is selected because it is immunogenic, a strong immune response to the vaccine in a diverse patient population is expected. In preferred embodiments, the carrier, like the hapten, must be sufficiently foreign to elicit a strong immune response to the vaccine. A conservative, but not essential, approach is to use a carrier to which most patients have not been exposed to avoid the phenomenon of carrier-induced epitope suppression. However, even if carrier-induced epitope suppression does occur, it is manageable as it has been overcome by dose changes (DiJohn et al. (1989) Lancet 1415-1418) and other protocol changes (Etlinger et al. (1990) Science 249:423-425), including the use of CTB (Stok et al. (1994) Vaccine 12:521-526). Vaccines which utilize carrier proteins to which patients are already immune are commercially available. Still further, carriers containing a large number of lysines are particularly suitable for conjugation according to the methods of the instant invention. In certain embodiments, therefore, the bacterial toxin carriers of the invention are modified so that their immunogenic properties are enhanced.

5.1.1 CTB as a Carrier

Cholera toxin is the enterotoxin produced by Vibrio cholerae and consists of five identical B subunits with each subunit having a molecular weight of 11.6 KDa (103 amino acids) and one A subunit of 27.2 KDa (230 amino acids) (Finkelstein (1988) Immunochem. Mol. Gen. Anal. Bac. Path. 85-102). The binding subunit, CTB, binds to ganglioside GM1 on the cell surface (Sixma et al. (1991) Nature 351:371-375; Orlandi et al. (1993) J. Biol. Chem. 268:17038-17044). CTA is the enzymatic subunit which enters the cell and catalyzes ADP-ribosylation of a G protein, constitutively activating adenylate cyclase (Finkelstein (1988) Immunochem. Mol. Gen. Anal. Bac. Path. pp. 85-102). In the absence of the A subunit, cholera toxin is not toxic.

In preferred embodiments of the invention, CTB is the bacterial toxin carrier. CTB is a highly immunogenic protein subunit capable of stimulating strong systemic and mucosal antibody responses (Lycke (1992) J. Immunol. 150:4810-4821; Holmgren et al. (1994) Am. J. Trop. Med. Hyg. 50:42-54; Silbart et al. (1988) J. Immun. Meth. 109:103-112; Katz et al. (1993) Infection Immun. 61:1964-1971). This combined IgA and IgG anti-hapten response is highly desirable in blocking, for example, cocaine or other substances for which is immunity is desired that are administered nasally or by inhalation, and in blocking nicotine or other substances that are absorbed in the mouth and lungs. In addition, CTB has already been shown to be safe for human use in clinical trials for cholera vaccines (Holmgren et al., supra; Jertborn et al. (1994) Vaccine 12:1078-1082; “The Jordan Report, Accelerated Development of Vaccines” 1993., NIAID, 1993). It is a discovery of this invention that hapten-carrier conjugates comprising CTB have even greater immunogenicity when CTB contains a signal peptide.

In one embodiment of the invention, the bacterial toxin carrier is CTB and its signal peptide is its endogenous signal peptide, or a fragment thereof. In one embodiment, the CTB signal peptide comprises the amino acid sequence Ala-Pro-Gly-Tyr-Ala-His-Gly. In other embodiments, the signal peptide comprises the amino acid sequence Gly-Tyr-Ala-His-Gly. In other embodiments, the signal peptide comprises the amino acid sequence Tyr-Ala-His-Gly. In other embodiments, the signal peptide comprises the amino acid sequence Ala-His-Gly. In other embodiments, the signal peptide comprises the amino acid sequence His-Gly. In other embodiments, the signal peptide comprises a single Gly residue. In some embodiments of the invention, the immunogenic compositions comprises a mixture of one or more of these peptides.

In certain embodiments, CTB bears a heterologous signal peptide.

5.1.1.1 CTB Preparation

Methods of making and using CTB as a carrier for incorporation into toxin-carrier conjugates are known. For example, see, U.S. Patent Application Publication No. 2005-0124061, published Jun. 9, 2005; U.S. Pat. No. 5,876,727, issued Mar. 2, 1999; and U.S. Pat. No. 5,760,184; each of which is incorporated herein by reference in its entirety. CTBs generated according to these methods, and the methods presented herein, may be modified so that it contains a signal peptide as described herein.

In a preferred embodiment, CTB is produced in Vibrio cholerae strain 213. See, for example, International Patent Application Publication No. WO 2005/042749, published May 12, 2005, which is incorporated by reference herein in its entirety.

In other embodiments, CTB is produced in E. coli (see, e.g., U.S. Patent Application Publication No. 2005-0124061, incorporated herein by reference in its entirety). Methods that enhance the isolation of forms of CTB that contain the signal peptide or a fragment thereof are preferred. The production of high level recombinant expression of CTB pentamers has been described (L′ hoir et al. (1990) Gene 89:47-52; Slos et al. (1994) Protein Exp. Purif. 5:518-526).

Native CTB is commercially available, and can be modified to have a signal peptide using standard techniques of protein engineering known to one of skill in the art. Recombinant CTB can be purified by ganglioside GM1 column affinity chromatography as described (Tayot et al. (1981) Eur. J. Biochem. 113:249-258). Recombinant CTB pentamer binds to ganglioside GM1 in an ELISA and reacts with pentamer-specific antibodies in Western blots and ELISA. Recombinant CTB is also available from other sources, such as SBL Vaccin AB.

The pentameric structure of CTB may be preferred for binding to ganglioside GM1. The pentamer is stable to SDS as long as the samples are not boiled, permitting pentamerization to be assessed by SDS-PAGE. Native CTB is a pentamer and is readily distinguishable from the denatured monomeric CTB on SDS-PAGE. Pentamer structure is maintained over a pH range from 4 to 9, which facilitates a variety of conjugation chemistries. The recombinant CTB initially expressed is monomeric. One way to obtain pentameric CTB is by making adjustments to express properly folded pentameric CTB. It has been found that cytoplasmic expression provides a much higher level of monomeric CTB. One skilled in the art is aware of methods of folding monomeric CTB into pentameric CTB (see, e.g., L′ hoir et al. (1990) Gene 89:47-52). An alternative to re-folding monomeric CTB to obtain pentameric CTB is periplasmic expression which results in pentameric recombinant CTB able to bind GM1-ganglioside by ELISA. One skilled in the art may find several approaches for obtaining pentameric recombinant CTB such as periplasmic expression with a leader (Slos et al., supra; Sandez et al. (1989) Proc. Nat'l. Acad. Sci. 86:481-485; Lebens et al. (1993) BioTechnol. 11:1574-1578) or post-translational refolding (L′ hoir et al., supra; Jobling et al. (1991) Mol. Microbiol. 5:1755-1767).

Amounts of recombinant CTB have been expressed and purified amounts which, once optimized, are produced in large fermentation batches. Processes for expressing and purifying recombinant protein are known in the art, for example, U.S. patent application Ser. No. 07/807,529. For example, CTB may be purified by affinity chromatography (Tayot et al. (1981) Eur. J. Biochem. 113:249-258), conjugated to cocaine or nicotine derivatives, and the conjugate may then be further purified. The purified CTB and the resulting conjugate are analyzed for purity and for maintenance of the pentameric structure of CTB. Techniques include SDS-PAGE, native PAGE, gel filtration chromatography, Western blotting, direct and GM1-capture ELISA, and competition ELISA with biotinylated CTB. Level of haptenation is measured by mass spectrometry, reverse phase HPLC and by analysis of the increase in UV absorbance resulting from the presence of the hapten. Both the solubility and the stability of the conjugate are optimized in preparation for full-scale formulation. Details of some of these analyses are given in the Examples.

Although the pentameric structure of CTB is a preferred carrier for practice of the present invention, and GM1 binding is an effective assay to determine that the pentameric form of CTB is present, the present invention is not limited to the use of the pentameric form of CTB. Other forms of CTB are contemplated (e.g., monomer, dimer, etc.) that may be manipulated for use in the invention. If a carrier other than the pentameric form of CTB is utilized, then one skilled in the art would use an appropriate assay to determine the presence and activity of the required carrier, e.g., the use of GM1 binding to determine the presence of the pentameric form of CTB).

Another useful CTB for use as a carrier is cholera toxin which provides improved mucosal response over CTB. It has been reported that the enzymatically active A subunit adjuvant enhances activity (Liang et al. (1988) J. Immunol. 141:1495-1501; Wilson et al. (1993) Vaccine 11:113-118; Snider et al. (1994) J. Immunol. 153:647).

5.2 Antigens for Use in the Conjugates of the Invention

The compositions and methods described herein are useful in inducing an immune response against a wide variety of antigens, for example antigens obtained or derived from diseased cells or tissues, or from human or animal pathogens, or from drugs of abuse. As used herein, an antigen on its own may or may not be immunogenic. Antigens that are immunogenic on their own and/or larger in size than a typical hapten are also contemplated for use in the conjugates of the invention, since addition of a signal peptide to a carrier will increase the immune response to the antigen.

As used herein, antigens also include the targets against which the conjugates of the invention will raise an immune response. For example, the antigen in an antigen-carrier conjugate may be a hepatitis virus epitope, and the antigen that the conjugate will raise an immune response against is the hepatitis virus itself.

For the purposes of the instant invention, the term “pathogen” is used in a broad sense to refer to a specific causative agent of a disease or condition, and includes any agent that provides a source of a molecule that elicits an immune response. Thus, pathogens include, but are not limited to, viruses, bacteria, fungi, protozoa, parasites, cancer cells and the like. Typically, the immune response is elicited by one or more peptide or carbohydrate antigens produced by the pathogen. Methods for identifying suitable antigens, obtaining and preparing such molecules, and then determining suitable dosages, assaying for suitable immunogenicity and treating with such antigens are well known in the art. See e.g., Plotkin et al. (1994) Vaccines, 2nd Edition, W. B. Saunders, Philadelphia, Pa. Non-limiting examples of sources for antigens that can be used to vaccinate vertebrate subjects, particularly, humans and non-human mammals, thus include viruses, bacteria, fungi, and other pathogenic organisms.

Viral antigens include, but are not limited to, those obtained or derived from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV). See, e.g., International Publication Nos. WO 89/04669; WO 90/11089; and WO 90/14436. The HCV genome encodes several viral proteins, including E1 and E2. See, e.g., Houghton et al. (1991) Hepatology 14:381-388. Genomic fragments containing sequences encoding these proteins, as well as antigenic fragments thereof, will find use in the present methods. Similarly, the coding sequence for the .delta.-antigen from HDV is known (see, e.g., U.S. Pat. No. 5,378,814).

In like manner, a wide variety of proteins from the herpesvirus family can be used as antigens in the present invention, including proteins derived from herpes simplex virus (HSV) types 1 and 2, such as HSV-1 and HSV-2 glycoproteins gB, gD and gH; antigens from varicella zoster virus (VZV), Epstein-Barr virus (EBV) and cytomegalovirus (CMV) including CMV gB and gH; and antigens from other human herpesviruses such as HHV6 and HHV7. (See, e.g. Chee et al. (1990) Cytomegaloviruses (J. K. McDougall, ed., Springer-Verlag, pp. 125-169; McGeoch et al. (1988) J. Gen. Virol. 69:1531-1574; U.S. Pat. No. 5,171,568; Baer et al. (1984) Nature 310:207-211; and Davison et al. (1986) J. Gen. Virol. 67:1759-1816.)

Human immunodeficiency virus (HIV) antigens, such as gp120 molecules for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. (1987) J. Virol. 61:570-578) and antigen-containing genomic fragments derived or obtained from any of these isolates will find use in the present invention. Furthermore, other immunogenic proteins derived or obtained from any of the various HIV isolates will find use herein, including fragments containing one or more of the various envelope proteins such as gp160 and gp41, gag antigens such as p24gag and p55gag, as well as proteins derived from the pol, env, tat, vif, rev, nef, vpr, vpu and LTR regions of HIV.

Antigens derived or obtained from other viruses will also find use herein, such as without limitation, antigens from members of the families Picornaviridae (e.g., polioviruses, rhinoviruses, etc.); Caliciviridae; Togaviridae (e.g., rubella virus, dengue virus, etc.); Flaviviridae; Coronaviridae; Reoviridae (e.g., rotavirus, etc.); Birnaviridae; Rhabdoviridae (e.g., rabies virus, etc.); Orthomyxoviridae (e.g., influenza virus types A, B and C, etc.); Filoviridae; Paramyxoviridae (e.g., mumps virus, measles virus, respiratory syncytial virus, parainfluenza virus, etc.); to Bunyaviridae; Arenaviridae; Retroviridae (e.g., HTLV-I; HTLV-II; HIV-1 (also known as HTLV-III, LAV, ARV, hTLR, etc.)), including but not limited to antigens from the isolates HIV Mb, HIV SF2, HIV LAV, HIV LAI, HIV MN); HIV-1 CM235, HIV-1 US4; HIV-2, among others; simian immunodeficiency virus (SIV); Papillomavirus, the tick-borne encephalitis viruses; and the like. See, e.g. Virology, 3rd Edition (W. K. Joklik ed. 1988); Fundamental Virology, 2nd Edition (B. N. Fields and D. M. Knipe, eds. 1991), for a description of these and other viruses.

In some contexts, it may be preferable that the selected viral antigens are obtained or derived from a viral pathogen that typically enters the body via a mucosal surface and is known to cause or is associated with human disease, such as, but not limited to, HIV (AIDS), influenza viruses (Flu), herpes simplex viruses (genital infection, cold sores, STDs), rotaviruses (diarrhea), parainfluenza viruses (respiratory infections), poliovirus (poliomyelitis), respiratory syncytial virus (respiratory infections), measles and mumps viruses (measles, mumps), rubella virus (rubella), and rhinoviruses (common cold).

Genomic fragments containing bacterial and parasitic antigens can be obtained or derived from known causative agents responsible for diseases including, but not limited to, Diphtheria, Pertussis, Tetanus, Tuberculosis, Bacterial or Fungal Pneumonia, Otitis Media, Gonorrhea, Cholera, Typhoid, Meningitis, Mononucleosis, Plague, Shigellosis or Salmonellosis, Legionnaire's Disease, Lyme Disease, Leprosy, Malaria, Hookworm, Onchoceriasis, Schistosomiasis, Trypamasomialsis, Leshmaniasis, Giardia, Amoebiasis, Filariasis, Borelia, and Trichinosis. Still further antigens can be obtained or derived from unconventional viruses such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

Specific pathogens can include M. tuberculosis, Chlamydia, N. gonorrhea, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pneumophila, Yersinia pestis, Streptococcus (types A and B), Pneumococcus, Meningococcus, Hemophilus influenza (type b), Toxoplasma gondic, Complylobacteriosis, Moraxella catarrhalis, Donovanosis, and Actinomycosis; fungal pathogens including Candidiasis and Aspergillosis; parasitic pathogens including Taenia, Flukes, Roundworms, Amebiasis, Giardiasis, Cryptosporidium, Schistosoma, Pneumocystis carinii, Trichomoniasis and Trichinosis. Thus, the present invention can also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneumoniae, E. coli, Bordetella pertussis, Bordetella parapertussis and brochiseptica.

In some embodiments, the antigen of interest can be an allergen. An “allergen” is an antigen which can initiate a state of hypersensitivity, or which can provoke an immediate hypersensitivity reaction in an individual already sensitized with the allergen. Allergens are commonly proteins or chemicals bound to proteins which have the property of being allergenic; however, allergens can also include organic or inorganic materials derived from a variety of man-made or natural sources such as plant materials, metals, ingredients in cosmetics or detergents, latexes, or the like. Classes of suitable allergens for use in the methods of the invention can include, but are not limited to, pollens, animal dander, grasses, molds, dusts, antibiotics, stinging insect venoms, find a variety of environmental (including chemicals and metals), drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Alternaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (dermalphagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common environmental allergens include metals (nickel and gold), chemicals (formaldehyde, trinitrophenol and turpentine), Latex, rubber, fiber (cotton or wool), burlap, hair dye, cosmetic, detergent and perfume allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der pI allergen (Hoyne et al. (1994) Immunology 83190-195), bee venom phospholipase A2 (PLA) (Akdis et al. (1996) J. Clin. Invest. 98:1676-1683), birch pollen allergen Bet v 1 (Bauer et al. (1997) Clin. Exp. Immunol. 107:536-541), and the multi-epitopic recombinant grass allergen rKBG8.3 (Cao et al. (1997) Immunology 90:46-51). These and other suitable allergens are commercially available and/or can be readily prepared as extracts following known techniques.

In certain other embodiments, the antigen of interest can be a tumor-specific antigen. For the purposes of the present invention, tumor-specific antigens include, but are not limited to, any of the various MAGEs (melanoma associated antigen E), including MAGE 1, MAGE 2, MAGE 3 (HLA-A1 peptide), MAGE 4, etc.; any of the various tyrosinases (HLA-A2 peptide); mutant Ras; mutant p53; and p97 melanoma antigen. Other tumor-specific antigens include the Ras peptide and p53 peptide associated with advanced cancers, the HPV 16/18 and E6/E7 antigens associated with cervical cancers, MUC1-KLH antigen associated with breast carcinoma, CEA (carcinoembryonic antigen) associated with colorectal cancer, gp100 or MART1 antigens associated with melanoma, and the PSA antigen associated with prostate cancer. The p53 gene sequence is known (see e.g., Harris et al. (1986) Mol. Cell. Biol. 6:4650-4656) and is deposited with GenBank under Accession No. M14694. Thus, the adjuvant compositions of the present invention can be used to carry out immunotherapeutic methods for treating cervical, breast, colorectal, prostate, lung cancers, and melanomas.

Antigens for use with the present invention can be obtained or produced using a variety of methods known to those of skill in the art. In particular, the antigens can be isolated directly from native sources, using standard purification techniques. Alternatively, the antigens can be produced recombinantly using known techniques. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985). Antigens for use herein may also be synthesized, based on described amino acid sequences, via chemical polymer syntheses such as solid phase peptide synthesis. Such methods are known to those of skill in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

If desired, polynucleotide sequences coding for the above-described antigens, can be obtained using recombinant methods, such as by screening cDNA and genomic libraries from cells expressing the gene, or by deriving the gene from a vector known to include the same. Furthermore, the desired gene can be isolated directly from cells and tissues containing the same, using standard techniques, such as phenol extraction and PCR of cDNA or genomic DNA. See, e.g., Sambrook et al., supra, for a description of techniques used to obtain and isolate DNA. Polynucleotide sequences can also be produced synthetically, rather than cloned.

5.2.1 Haptens for Use in the Immunogenic Compositions of the Invention

An antigen of the invention can be a hapten. In one embodiment, a “hapten” as used in the invention is a low-molecular-weight organic compound that reacts specifically with an antibody and which is incapable of inciting an immune response by itself but is immunogenic when complexed to a T cell epitope-containing carrier forming a hapten-carrier conjugate. In other embodiments, the hapten is poorly immunogenic by itself. Further, the hapten is characterized as the specificity-determining portion of the hapten-carrier conjugate, that is, it is capable of reacting with an antibody specific to the hapten in its free state. In some embodiments, in a non-immunized subject, there is an absence of formation of antibodies to the hapten. In some embodiments, in a non-immunized subject, there may be a low level of antibodies to the hapten, or a level of antibodies to the hapten for which an increase in the immune response to the hapten is desired. In the instant invention, in certain embodiments, the term hapten shall include the concept of a more specific drug/hapten which is a drug, an analog of a portion of the drug, or drug derivative. The immunogenic composition, or, in some embodiments, the vaccine, when initially administered will give rise to a “desired measurable outcome.” Initially, the desired measurable outcome is the production of a high titer of anti-hapten antibodies (approximately 0.1 mg/ml to 1 mg/ml or greater of specific antibody in the serum). However, manipulation of the dosage regimen suitable for the individual gives and maintains a sustained desired therapeutic effect. The “desired therapeutic effect” is the neutralization of a sufficient fraction of free hapten to reduce or eliminate the pharmacological effects of the hapten (e.g., nicotine or cocaine) within a therapeutically acceptable time frame by anti-hapten antibodies specific for the hapten upon a subsequent exposure to the hapten. Determining the therapeutically acceptable time frames for how long it takes to get a sufficient antibody response to a given hapten and how-long is that antibody response is maintained thereto are achieved by those skilled in the art by assessing the characteristics of the subject to be immunized, hapten (e.g., drug of abuse) to be neutralized, as well as the mode of administration. Using this and other immunization protocols as a model, one skilled in that art would expect the immunity or the period of protection to last several months, up to more than one year.

One aspect of achieving a conjugate of the instant invention involves modifying the hapten sufficiently to render it capable of being conjugated or joined to a carrier while maintaining enough of the structure so that it is recognized as free state hapten (for example, as free cocaine or nicotine). It is essential that a vaccinated individual has antibodies which recognize free hapten (e.g., cocaine or nicotine). Radioimmunoassay and competition ELISA assay experiments can measure antibody titers to free hapten. Antibodies of interest are hapten-specific antibodies and, in some embodiments, are cocaine-specific antibodies or nicotine-specific antibodies. It should be recognized that principles and methods used to describe the preferred embodiments may be extended from this disclosure to a wide range of hapten-carrier conjugates useful in the treatment of a variety of diseases, conditions, or drug addictions and toxic responses.

Various haptens may be used in the practice of the invention. In some embodiments, the hapten is an antigen selected from the antigens in Section 5.2. above. In some embodiments, the hapten is a drug, such as, for example,

Hallucinogens, for example mescaline and LSD;

Cannabinoids, for example THC;

Stimulants, for example amphetamines, cocaine, phenmetrazine, methylphenidate;

Nicotine;

Depressants, for example, nonbarbiturates (e.g. bromides, chloral hydrate etc.), methaqualone, barbiturates, diazepam, flurazepam, phencyclidine, and fluoxetine;

Opium and its derivatives, for example, heroin, methadone, morphine, meperidine, codeine, pentazocine, and propoxyphene; and

“Designer drugs” such as “ecstasy.”

5.3 Methods of Preparing Antigen-Carrier and Hapten-Carrier Conjugates

Preparation of the Conjugates of the Instant Invention is Exemplified in this Section using haptens derived from cocaine and cocaine metabolites, primarily derivatives of norcocaine, benzoyl ecgonine and ecgonine methyl ester, and a variety of carriers, including recombinant CTB (rCTB). These methods can be adapted for use with any other hapten, as exemplified in the next section with nicotine.

The length and nature of the hapten-carrier linkage is such that the hapten is displaced a sufficient distance from the carrier domain to allow its optimal recognition by the antibodies initially raised against it. The length of the linker is optimized by varying the number of —CH₂ groups which are strategically placed within a “branch” selected from the group consisting of:
CJ 0 Q
CJ 1 (CH₂)ₙQ
CJ 1.1 CO₂Q
CJ 1.2 COQ
CJ 1.3 OCH₃
CJ 2 OCO(CH₂)ₙQ
CJ 2.1 OCOCH=Q
CJ 2.2 OCOCH(O)CH₂
CJ 2.3 OCO(CH₂)ₙCH(O)CH₂
CJ 3 CO(CH₂)ₙCOQ
CJ 3.1 CO(CH₂)ₙCNQ
CJ 4 OCO(CH₂)ₙCOQ
CJ 4.1 OCO(CH₂)ₙCNQ
CJ 5 CH₂OCO(CH₂)ₙCOQ
CJ 5.1 CH₂OCO(CH₂)ₙCNQ
CJ 6 CONH(CH₂)ₙQ
CJ 7 Y(CH₂)ₙQ
CJ 7.1 CH₂Y(CH₂)ₙQ
CJ 8 OCOCH(OH)CH₂Q
CJ 8.1 OCO(CH₂)ₙCH(OH)CH₂Q
CJ 9 OCOC₆H₅

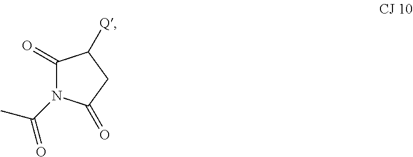

wherein Q′ is a modified protein; and
CJ 11 YCO(CH₂)ₙCOQ.

(See also U.S. Patent Application Publication No. 2005-0124061, incorporated herein by reference in its entirety). With regard to the above branches, n is an integer preferably selected from about 1 to about 20, more particularly about 3 to about 6; Y is preferably selected from the group consisting of S, O, and NH; and Q is preferably selected from the group consisting of

(i) —H;

(ii) —OH;

(iii) —CH₂;

(iv) —CH;

(iv a) —OCH₃;

(v) —COOH;

(vi) a halogen;

(vii) an activated ester or esters, such as 2-nitro-4-sulfophenyl ester and N-oxysuccinimidyl ester;

(viii) a group or groups reactive toward the carrier, such as a mixed anhydride, acyl halide, acyl azide, alkyl halide, N-maleimide, imino ester, isocyanate, and isothiocyanate;

(ix) the carrier; and

(x) another “branch” identified by its “CJ” reference number.

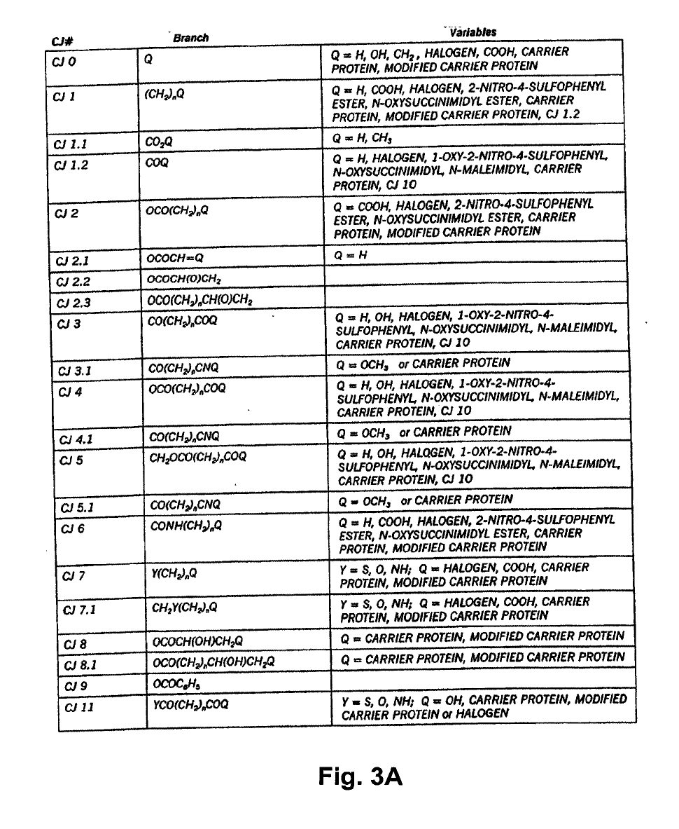

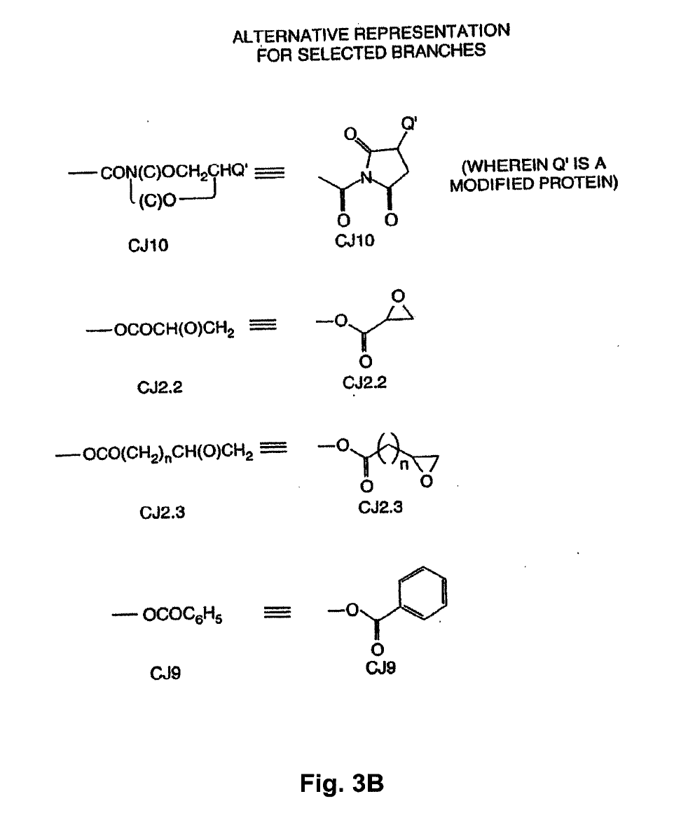

A T cell epitope containing carrier may be modified by methods known to those skilled in the art to facilitate conjugation to the hapten (e.g., by thiolation). For example with 2-iminothiolane (Traut's reagent) or by succinylation, etc. For simplicity, (CH₂)_{Q}, where Q=H, may be referred to as (CH₃), methyl or Me, however, it is understood that it fits into the motif as identified in the “branches” as shown in FIGS. 3a and 3b. Further abbreviations of commercially obtainable compounds used herein include:

BSA=bovine serum albumin

DCC=Dicyclohexylcarbodiimide

DMF=N,N-Dimethylformamide

EDC (or EDAC)=N-Ethyl-N′-(3-(dimethylamino) propyl)carbodiimide hydrochloride

EDTA=Ethylenediamine tetraacetic acid, disodium salt

HATU=O-(7-Azabenzotriazol-1-yl)-1,1,3,3-tetramethyluronium hexafluorophosphate

NMM=N-Methylmorpholine

HBTU=2-(1H-Benzotriazole-1-yl)-1,1,3,3-tetramethyluronium hexafluorophosphate

TNTU=2-(5-Norbornene-2,3-dicarboximido)-1,1,3,3-tetramethyluronium tetrafluoroborate

PyBroP®=Bromo-tris-pyrrolidino-phosphonium hexafluorophosphate

HOBt=N-Hydroxybenzotriazole

Further the IUPAC nomenclature for several named compounds are:

Norcocaine:

3β-(Benzoyloxy)-8-azabicyclo[3.2.1]octane-2β-carboxylic acid methyl ester

Benzoyl Ecgonine:

3β-(Benzoyloxy)-8-methyl-8-azabicyclo[3.2.1]octane-2β-carboxylic acid

Cocaine:

3β-(Benzoyloxy)-8-methyl-8-azabicyclo[3.2.1]octane-2β-carboxylic acid methyl ester

Ecgonine Methyl Ester:

3β-(Hydroxy)-8-methyl-8-azabicyclo[3.2.1]octane-2β-carboxylic acid methyl ester

Nicotine

1-Methyl-2-(3-pyridyl)pyrrolidine

Cotinine

N-Methyl-2-(3-pyridyl)-5-pyrrolidone

5.4 Preparation of Nicotine Conjugates

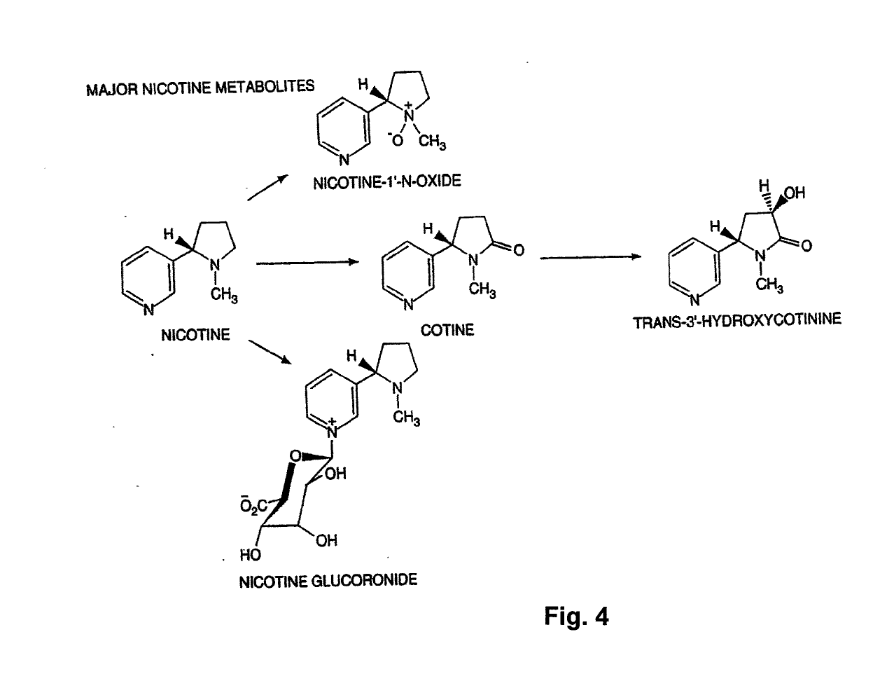

The novel nicotine-carrier conjugates of the present invention are derived from nicotine and nicotine metabolites. FIG. 4 shows a representation of nicotine and some of its derivatives and metabolites.

In addition to adapting the methods described above for the preparation of nicotine-carrier conjugates, precursors of nicotine-carrier conjugates can be synthesized by selectively alkylating the pyridine nitrogen in (S)-(−)-nicotine in anhydrous methanol, with ethyl 3-bromobutyrate, 5-bromovaleric acid, 6-bromohexanoic acid or 8-bromooctanoic acid respectively. The products of these reactions are conjugated to a carrier protein using HATU.

In another embodiment, to a solution of nornicotine (50 mmol) in methylene chloride is added triethylamine (75 mmol), followed by succinic anhydride (100 mmol). The solution is heated at reflux for 18 hours. The reaction mixture is washed sequentially with 10% aqueous hydrochloric acid, saturated sodium bicarbonate solution, brine and water. After drying (MgSO₄) and removal of the solvents under reduced pressure, the residue is purified using silica gel flash chromatography to furnish the desired product.

In a further embodiment, the succinylated nornicotine is used to synthesize the nicotine conjugate. To a solution of succinylated nornicotine (5 μmol) in DMF (0.1 ml), diisopropylethylamine (10 mmol) is added followed by HATU (5.5 μmol). After 10 minutes, the pale yellow solution is added dropwise to a solution of either HEL or BSA (500 μg) in 0.1 M sodium borate buffer at pH 8.8 (0.9 ml) and the mixture stirred for 18 hours at ambient temperature. The pH of the conjugate solution is adjusted to pH 7.0 by careful addition of 0.1 M aqueous hydrochloric acid, followed by purification by dialysis against PBS. The dialysate is filtered through a 0.2 μm filter and the level of haptenation measured by mass spectral analysis or UV absorbance. These and other methods for making nicotine-carrier conjugates are described below.

5.4.1 Method A: N′-Butyric Acid Adduct of (S)-Nicotine

To a solution of (S)-nicotine (0.031 moles) in anhydrous methanol (50 ml) at ice-water temperature under argon, ethyl-4-bromobutyrate (0.0341 moles) is added dropwise over 10 minutes. The resulting orange colored solution is allowed to warm to ambient temperature and stirred for 18 hours. The solvents are removed under reduced pressure leaving a brown residue which is precipitated with hexane to give an analytically pure sample of the desired ester.

The ester (36 mg) is dissolved in methanol (3 ml) and 1M sodium hydroxide solution (5 ml) and stirred for 18 hours at ambient temperature. The solvents are removed under reduced pressure and the residue dissolved in 10% hydrochloric acid and extracted with ethyl acetate. Following drying (MgSO₄) the solvents are removed under reduced pressure to yield the desired compound.

5.4.2 Method B: N′-Valeric Acid Adduct of (S)-Nicotine

To a solution of (S)-nicotine (0.031 moles) in anhydrous methanol (50 ml) at ice-water temperature under argon, 1-bromovaleric acid (0.0341 moles) is added dropwise over 10 minutes. The resulting orange colored solution is allowed to warm to ambient temperature and stirred for 18 hours. The solvents are removed under reduced pressure leaving a brown residue which are precipitated with hexane to give an analytically pure sample of the desired compound.

5.4.3 Method C: N′-Hexanoic Acid Adduct of (S)-Nicotine

To a solution of (S)-nicotine (0.031 moles) in anhydrous methanol (50 ml) at ice-water temperature under argon, 1-bromohexanoic acid (0.0341 moles) is added dropwise over 10 minutes. The resulting orange colored solution is allowed to warm to ambient temperature and stirred for 18 hours. The solvents are removed under reduced pressure leaving a brown residue which are precipitated with hexane to give an analytically pure sample of the desired compound.

5.4.4 Method D: N′-Octanoic Acid Adduct of (S)-Nicotine

To a solution of (S)-nicotine (0.031 moles) in anhydrous methanol (50 ml) at ice-water temperature under argon, the appropriate 1-bromooctanoic acid (0.0341 moles) is added dropwise over 10 minutes. The resulting orange colored solution is allowed to warm to ambient temperature and stirred for 18 hours. The solvents are removed under reduced pressure leaving a brown residue which is precipitated with hexane to give an analytically pure sample of the desired compound.

5.4.5 Other Methods

In certain embodiments, to a solution of the appropriate N′-alkanoic acid analog of nicotine (6.27×10⁻⁵ moles) in DMF (1.6 ml), DIEA (1.25×10⁻⁴ moles) and HATU (7.53×10⁻⁵ moles) are added. After 10 minutes at ambient temperature, the pale yellow solution is added to either HEL or BSA (16.5 mg) in 0.1M sodium bicarbonate, pH 8.3 (14.4 ml) and stirred for 18 hours. The conjugate solution is purified by dialysis against PBS at 4° C. overnight. The conjugates are analyzed using laser desorption mass spectral analysis to determine the number of haptens.

In a preferred embodiment in which CTB is the carrier, to a solution of the appropriate N′-alkanoic acid analog of nicotine (6.27×10⁻⁵ moles) in DMF (1.6 ml), DIEA (1.25×10⁻⁴ moles) and HATU (7.53×10⁻⁵ moles) are added. After 10 minutes at ambient temperature, the pale yellow solution is added to rCTB (16.6 mg) in 0.1M sodium bicarbonate, pH 8.3 (14.4 ml) and stirred for 18 hours. The conjugate solution is purified by dialysis against PBS at 4° C. overnight. The conjugates are analyzed using laser desorption mass spectral analysis to determine the number of haptens.

5.5 Immunogenic Compositions and Methods for their Use

The present invention provides immunogenic compositions comprising a hapten-carrier conjugate, in which the carrier is a bacterial toxin that has a signal peptide. In one embodiment, the hapten is nicotine or a nicotine derivative. In another embodiment, the hapten is cocaine or cocaine derivative. In one embodiment, the bacterial toxin carrier is CTB with its endogenous signal peptide or fragment or fragments thereof. The immunogenic compositions of the invention comprise such toxin-carrier conjugates and optionally a physiological carrier or excipient. The invention provides methods for producing such immunogenic compositions, comprising producing the bacterial toxin carrier in a system that allows isolation of the carrier with a signal peptide or fragment thereof or adding the signal peptide after isolation of the carrier and then conjugating it to the hapten. In some embodiments, the hapten is a proteinaceous substance, in which case the conjugate can be produced recombinantly by propagation in a substrate. The invention provides methods of inducing an immune response, comprising administering to a subject an effective amount of an immunogenic composition of the invention. The invention provides methods of preventing, managing and/or treating a disease or condition, including drug addiction, comprising administering an effective amount of an immunogenic composition of the invention. In some embodiments, the drug addiction to be prevented, managed and/or treated is cocaine addiction. In other embodiments, the drug addiction to be prevented, managed and/or treated is nicotine addiction.

As defined herein, an immunogenic composition of the invention is able to induce an immune response in a cell, tissue, organ, and/or subject or patient. As used herein, the terms “subject” or “patient” are used interchangeably. As used herein, the terms “subject” and “subjects” refer to an animal (e.g., birds, reptiles, and mammals), preferably a mammal including a non-primate (e.g., a camel, donkey, zebra, cow, pig, horse, goat, sheep, cat, dog, rat, and mouse) and a primate (e.g., a monkey, chimpanzee, and a human), and most preferably a human. In certain embodiments, the subject or patient has a drug addiction. In certain embodiments, the subject or patient is at risk for developing or re-developing a drug addiction.

In some embodiments of the invention, the immunogenic composition is a “vaccine,” i.e., is for administration to a subject or patient.

In some embodiments of the invention, the immunogenic compositions induce an immune response from the adaptive immune system, such as a B cell response and/or a T cell response. In some embodiments, the immune response induced by the immunogenic composition is an antibody response. In some embodiments, the immunogenic composition induces a humoral immune response, such as an interferon response and/or an interleukin response, e.g., an interleukin-4 response. In some embodiments, the immunogenic composition induces one or more types of immune response but not another immune response. In certain embodiments, the immunogenic composition induces a combination of immune responses. Moreover, in some embodiments, the immunogenic compositions can induce a robust IFN response which has other biological consequences in vivo, affording protection against subsequent diseases or conditions or concurrent diseases or conditions. In some embodiments, the immunogenic compositions can induce a robust TNFα or interleukin response which has other biological consequences in vivo, affording protection against subsequent diseases or conditions or concurrent diseases or conditions.

In certain embodiments, the immune response induced by an immunogenic composition of the invention, comprising a conjugate in which the carrier is a signal peptide containing bacterial toxin, is increased 5-10%, 10-20%, 20-30%, 30-40%, 40-50%, 50-60%, 60-70%, 70-80%, 80-90%, 90-100% or more compared to a subject (host) or host cell administered a placebo or other negative control. In certain embodiments, the immune response induced by an immunogenic composition of the invention, comprising a conjugate in which the carrier is a signal peptide containing bacterial toxin, is increased approximately 1 to approximately 100 fold, approximately 5 to approximately 80 fold, approximately 20 to approximately 80 fold, approximately 1 to approximately 10 fold, or approximately 1 to approximately 5 fold, or approximately 40 to approximately 80 fold, or 1, 2, 3, 4, 5, 7, 10, 15, 20, 25, 30, 35, 40, 45, 50, 55, 60, 65, 70, 75, 80, 85, 90, 95 or 100 fold compared to a subject (host) or host cell administered a placebo or other negative control.

In certain embodiments, the immune response induced by an immunogenic composition of the invention, comprising a conjugate in which the carrier is a signal peptide containing bacterial toxin, is increased 5-10%, 10-20%, 20-30%, 30-40%, 40-50%, 50-60%, 60-70%, 70-80%, 80-90%, 90-100% or more compared to a subject (host) or host cell administered a conjugate that lacks the signal peptide. In certain embodiments, the immune response induced by an immunogenic composition of the invention, comprising a conjugate in which the carrier is a signal peptide containing bacterial toxin, is increased approximately 1 to approximately 100 fold, approximately 5 to approximately 80 fold, approximately 20 to approximately 80 fold, approximately 1 to approximately 10 fold, or approximately 1 to approximately 5 fold, or approximately 40 to approximately 80 fold, or 1, 2, 3, 4, 5, 7, 10, 15, 20, 25, 30, 35, 40, 45, 50, 55, 60, 65, 70, 75, 80, 85, 90, 95 or 100 fold compared to a subject (host) or host cell administered a conjugate that lacks the signal peptide.

In certain embodiments, the antibody response induced by an immunogenic composition of the invention, comprising a conjugate in which the carrier is a signal peptide containing bacterial toxin, is increased 5-10%, 10-20%, 20-30%, 30-40%, 40-50%, 50-60%, 60-70%, 70-80%, 80-90%, 90-100% or more compared to a subject (host) or host cell administered a conjugate that lacks the signal peptide. In certain embodiments, the antibody response induced by an immunogenic composition of the invention, comprising a conjugate in which the carrier is a signal peptide containing bacterial toxin, is increased approximately 1 to approximately 100 fold, approximately 5 to approximately 80 fold, approximately 20 to approximately 80 fold, approximately 1 to approximately 10 fold, or approximately 1 to approximately 5 fold, or approximately 40 to approximately 80 fold, or 1, 2, 3, 4, 5, 7, 10, 15, 20, 25, 30, 35, 40, 45, 50, 55, 60, 65, 70, 75, 80, 85, 90, 95 or 100 fold compared to a subject (host) or host cell administered a conjugate that lacks the signal peptide.

In certain embodiments, the interferon response or interleukin response, preferably the IL-4 response, induced by an immunogenic composition of the invention, comprising a conjugate in which the carrier is a signal peptide containing bacterial toxin, is increased 5-10%, 10-20%, 20-30%, 30-40%, 40-50%, 50-60%, 60-70%, 70-80%, 80-90%, 90-100% or more compared to a subject (host) or host cell administered a conjugate that lacks the signal peptide. In certain embodiments, the interferon response or interleukin response, preferably the IL-4 response induced by an immunogenic composition of the invention, comprising a conjugate in which the carrier is a signal peptide containing bacterial toxin, is increased approximately 1 to approximately 100 fold, approximately 5 to approximately 80 fold, approximately 20 to approximately 80 fold, approximately 1 to approximately 10 fold, or approximately 1 to approximately 5 fold, or approximately 40 to approximately 80 fold, or 1, 2, 3, 4, 5, 7, 10, 15, 20, 25, 30, 35, 40, 45, 50, 55, 60, 65, 70, 75, 80, 85, 90, 95 or 100 fold compared to a subject (host) or host cell administered a conjugate that lacks the signal peptide.

In some embodiments, the immunogenic compositions of the present invention comprise an effective amount of a conjugate of the invention, and a pharmaceutically acceptable carrier. The term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical formulation is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration. The particular composition may also depend on whether the mutant virus is live or inactivated.

The immunogenic compositions of the invention may be administered to a naïve subject, i.e., a subject that does not have a disease, condition, drug addiction, or has not been and is not currently infected with an infectious agent. The immunogenic compositions of the invention may be administered to a naïve subject, i.e., a subject that does not have a disease, condition, drug addiction, or has not been and is not currently infected with an infectious agent, but is predisposed to acquiring such disease, condition, drug addiction, or infection. The immunogenic compositions of the invention may also be administered to a subject that has and/or has had a disease, condition, drug addiction, or infection.

Many methods may be used to introduce the immunogenic compositions, e.g., vaccine formulations described herein. These include but are not limited to intranasal, intratracheal, oral, intradermal, intramuscular, intraperitoneal, intravenous, conjunctival and subcutaneous routes. As an alternative to parenteral administration, the invention also encompasses, routes of mass administration for agricultural purposes such as via drinking water or in a spray. It may be preferable to introduce the mutant virus of the invention via the natural route of administration or infection of the agent against which the immunogenic composition is targeted.

In certain embodiments, an immunogenic composition of the invention does not result in complete protection or cure (i.e., from drug addiction), but results in a lower level of addiction compared to an untreated subject. Benefits include, but are not limited to, reduced severity of symptoms of the disease or condition and a reduction in the duration of the disease or condition

In certain embodiments, an immunogenic composition of the invention is used to protect against a disease or condition (e.g., an infection or drug addiction) in naïve subjects.

The prophylactic and/or therapeutic effect of the immunogenic compositions of the invention are based, in part, upon achieving or inducing an immune response (e.g., a humoral immune response or adaptive immune response). In one aspect, the immunogenic compositions induce a detectable serum titer of an antibody against an antigen or hapten in either the subject or an animal model thereof (e.g. mouse, rat, pig, goat, sheep or canine model). The serum titer of an antibody can be determined using techniques known to one of skill in the art, e.g., immunoassays such as ELISAs. In a specific embodiment, the antibodies generated by administering an immunogenic composition of the invention are neutralizing antibodies.

In one embodiment, administration of an immunogenic composition of the invention to a subject or animal model thereof results in a serum titer of about 1 μg/ml, about 2 μg/ml, about 5 μg/ml, about 6 μg/ml, about 10 μg/ml, about 15 μg/ml, about 20 μg/ml, about 25 μg/ml, about 50 μg/ml, about 75 μg/ml, about 100 μg/ml, about 125 μg/ml, about 150 μg/ml, about 175 μg/ml, about 200 μg/ml, about 225 μg/ml, about 250 μg/ml, about 275 μg/ml, about 300 μg/ml, about 325 μg/ml, about 350 μg/ml, about 375 μg/ml, or more of an antibody that specifically binds to the hapten or antigen. In certain preferred embodiments, the serum titer is 100 μg/ml or more. In some embodiments, administration of an immunogenic composition of the invention results in a plasma titer of 100 μg/ml to 1 mg/ml or more, preferably more than about 500 μg/ml. The immune response may be determined in the subject or in a animal model, which response is then correlated or extrapolated to a predicted response in the subject, e.g., a human or livestock, such as a pig, sheep, goat, or cow.

In one embodiment, the present invention provides methods for preventing, treating, managing, or ameliorating at least one disease or condition (e.g., a drug addiction or viral infection) in a subject, the methods comprising administering to said subject an effective amount of an immunogenic composition comprising a conjugate of the invention. In some embodiments, the dose of the immunogenic composition administered to the subject or animal model is about 10-20 μg. In some embodiments, the dose of the immunogenic composition administered to the subject or animal model is about 75-100 μg. In some embodiments, the dose of the immunogenic composition administered to the subject or animal model is about 500-1000 μg.

The present invention provides methods for preventing, treating, managing, or ameliorating at least one disease or condition (e.g., a drug addiction or viral infection) in a subject, the methods comprising administering to said subject an effective amount of an immunogenic composition comprising a conjugate of the invention, wherein the effective amount is the amount that results in a reduction in mortality, reduction in hospitalization, reduction in the severity of the disease or condition and/or reduction in the clinical symptoms of the disease or condition relative to a subject not administered the immunogenic composition of the invention or administered a hapten-bacterial toxin conjugate in which the bacterial toxin lacks a signal peptide. In certain preferred embodiments the subject is a human. In certain embodiments, the subject is a mouse or a rat.

The amount of the immunogenic composition of the invention which will be effective in the treatment, prevention and/or amelioration of a particular disease or condition will depend on the nature of the disease, and can be determined by standard clinical techniques. In addition, in vitro assays may optionally be employed to help identify optimal dosage ranges. The precise dose to be employed in the composition will also depend on the route of administration, and the seriousness of the disease or disorder, and should be decided according to the judgment of the practitioner and each subject's circumstances. However, suitable dosage ranges for administration are generally about 10-20 μg, 20-50 μg, 50-75 μg, 75-100 μg, 100-200 μg, 200-300 μg, 300-400 μg, 400-500 μg, 500-600 μg, 600-700 μg, 700-800 μg, 800-900 μg, or 900-1000 μg, or more. Effective doses may be extrapolated from dose response curves derived from in vitro or animal model test systems.

In various embodiments, the immunogenic compositions of the invention or antibodies generated by them are administered to a subject in combination with one or more other therapies for the prevention or treatment of at least one disease or condition. In certain embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient or subject visit. Non-limiting examples of agents that can be administered in combination with an immunogenic composition of the invention or an antibody generated by the composition of the invention are found below.

5.5.1 Uses of the Immunogenic Compositions of the Invention

In some embodiments, the compositions of the invention are useful in the treatment or prevention of a disease, condition, or infection. In certain embodiments, the condition is a drug addiction or drug allergy. In certain specific embodiments, the drug is nicotine. In other embodiments, the drug is cocaine. In preferred embodiments, the immunogenic compositions of the invention exhibit specificity for a hapten compared to cellular components and/or compared to the carrier. In another embodiment, the immunogenic compositions of the invention exhibits low cytotoxicity in eukaryotic cells, preferably mammalian cells.

In one embodiment, an immunogenic composition of the invention reduces or inhibits a drug addiction, dependence, or allergy. In a specific embodiment, the immunogenic composition eliminates or reduces the drug addiction or drug dependence by 75%, 80%, 85%, 90%, 95%, 98%, 99%, 75-99.5%, 85-99.5%, or 90-99.8% in a subject as determined by an assay described herein or known to one of skill in the art. Accordingly, the immunogenic compositions of the invention are useful in methods of preventing, treating and/or managing drug addiction or drug dependence. In a particular embodiment, an immunogenic composition of the invention is useful in preventing, treating and/or managing a disease or condition that exhibits resistance to other treatments.

In certain embodiments, an immunogenic composition of the invention inhibits or reduces the circulation in the bloodstream a drug or antigen against which it is targeted by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured by a standard assay known to one of skill in the art, or an assay described herein.

In some embodiments, an immunogenic composition of the invention inhibits or reduces the penetration of the drug or antigen against which it is targeted from one organ, tissue or cell to another organ, tissue or cell as measured using a standard assay known to one of skill in the art, or an assay described herein. In some embodiments, an immunogenic composition of the invention inhibits or reduces the ability of a drug, such as cocaine or nicotine, to enter the brain by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% as measured using a standard assay known to one of skill in the art, or an assay described herein.

5.5.2 Prophylactic and Therapeutic Methods

The present invention provides methods of preventing, treating and/or managing a disease or condition, such as a drug addiction, said methods comprising administering to a subject in need thereof one or more immunogenic compositions of the invention. In one embodiment, the invention provides a method of preventing, treating/and or managing a cocaine addiction or a nicotine addiction.

The invention also provides methods of preventing, treating and/or managing a disease or condition, said methods comprising administering to a subject in need thereof one or more of the immunogenic compositions of the invention, and one or more other therapies (e.g., prophylactic or therapeutic agents). In a specific embodiment, the other therapies are currently being used, have been used or are known to be useful in the prevention, treatment and/or management of the disease or condition. Non-limiting examples of such prophylactic or therapeutic methods are provided below.

The combination therapies of the invention can be administered sequentially or concurrently. In one embodiment, the combination therapies of the invention comprise a compound of the invention and at least one other therapy which has the same mechanism of action. In another embodiment, the combination therapies of the invention comprise a compound of the invention and at least one other therapy which has a different mechanism of action than the compound.

In a specific embodiment, the combination therapies of the present invention improve the prophylactic and/or therapeutic effect of an immunogenic composition of the invention by functioning together with immunogenic composition to have an additive or synergistic effect. In another embodiment, the combination therapies of the present invention reduce the side effects associated with each therapy taken alone.

The prophylactic or therapeutic agents of the combination therapies can be administered to a subject in the same immunogenic composition. Alternatively, the prophylactic or therapeutic agents of the combination therapies can be administered concurrently to a subject in separate immunogenic compositions. The prophylactic or therapeutic agents may be administered to a subject by the same or different routes of administration.

In a specific embodiment, an immunogenic composition comprising one or more conjugates of the invention and a pharmaceutically acceptable carrier or excipient is administered to a subject, preferably a human, to prevent, treat and/or manage a drug addiction. In accordance with the invention, the immunogenic compositions may also comprise one or more other prophylactic or therapeutic agents. In a specific embodiment, the other prophylactic or therapeutic agents are currently being used, have been used or are known to be useful in the prevention, treatment and/or management of the drug addiction or a symptom or condition related to it, e.g., a psychiatric or psychological condition.

An immunogenic composition of the invention may be used as any line of therapy, e.g., a first, second, third, fourth or fifth line therapy, for a disease or condition. In some embodiments, the subject administered an immunogenic composition of the invention in accordance with the invention has not received a therapy prior to the administration of the immunogenic composition of the invention. In other embodiments, the subject administered an immunogenic composition of the invention in accordance with the invention has received a therapy prior to administration of the immunogenic composition of the invention. In some embodiments, the subject administered an immunogenic composition of the invention in accordance with the invention was refractory to a prior therapy or experienced adverse side effects to the prior therapy or the prior therapy was discontinued due to unacceptable levels of toxicity to the subject.

The invention provides methods for treating and/or managing a disease or condition, e.g., a drug addiction, in a subject refractory to conventional therapies for such a condition, the methods comprising administering to said subject a dose of a prophylactically or therapeutically effective amount of an immunogenic composition of the invention.

5.5.2.1 Agents Useful in Combination with the Conjugates or Immunogenic Compositions of the Invention

Therapeutic or prophylactic agents that can be used in combination with an immunogenic composition of the invention for the prevention, treatment and/or management of a disease or condition include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such agents include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, and non-steroidal anti-inflammatory drugs (e.g., aspirin, ibuprofen, diclofenac, and COX-2 inhibitors), pain relievers, anti-psychotics, anti-depressants, anti-anxiety drugs, anti-epileptics, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythomycin, penicillin, mithramycin, and anthramycin (AMC)).

Any therapy which is known to be useful, or which has been used or is currently being used for the prevention, management, and/or treatment of the disease or condition, or a disease or condition associated with the disease or condition, can be used in combination with an immunogenic composition of the invention in accordance with the invention described herein. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996 for information regarding therapies (e.g., prophylactic or therapeutic agents) which have been or are currently being used for preventing, treating and/or managing various diseases or conditions.

Antibacterial agents, including antibiotics, that can be used in combination with an immunogenic composition of the invention include, but are not limited to, aminoglycoside antibiotics, glycopeptides, amphenicol antibiotics, ansamycin antibiotics, cephalosporins, cephamycins oxazolidinones, penicillins, quinolones, streptogamins, tetracyclins, and analogs thereof.

In a specific embodiment, an immunogenic composition of the invention is used in combination with other protein synthesis inhibitors, including but not limited to, streptomycin, neomycin, erythromycin, carbomycin, and spiramycin.

In one embodiment, the antibacterial agent is selected from the group consisting of ampicillin, amoxicillin, ciprofloxacin, gentamycin, kanamycin, neomycin, penicillin G, streptomycin, sulfanilamide, and vancomycin. In another embodiment, the antibacterial agent is selected from the group consisting of azithromycin, cefonicid, cefotetan, cephalothin, cephamycin, chlortetracycline, clarithromycin, clindamycin, cycloserine, dalfopristin, doxycycline, erythromycin, linezolid, mupirocin, oxytetracycline, quinupristin, rifampin, spectinomycin, and trimethoprim

Additional, non-limiting examples of antibacterial agents for use in combination with an immunogenic composition of the invention include the following: aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), folic acid analogs (e.g., trimethoprim), glycopeptides (e.g., vancomycin), lincosamides (e.g., clindamycin, and lincomycin), macrolides (e.g., azithromycin, carbomycin, clarithomycin, dirithromycin, erythromycin, and erythromycin acistrate), monobactams (e.g., aztreonam, carumonam, and tigemonam), nitrofurans (e.g., furaltadone, and furazolium chloride), oxacephems (e.g., flomoxef, and moxalactam), oxazolidinones (e.g., linezolid), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o benethamine, penicillin 0, penicillin V, penicillin V benzathine, penicillin V hydrabamine, penimepicycline, and phencihicillin potassium), quinolones and analogs thereof (e.g., cinoxacin, ciprofloxacin, clinafloxacin, flumequine, grepagloxacin, levofloxacin, and moxifloxacin), streptogramins (e.g., quinupristin and dalfopristin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), and tetracyclines (e.g., apicycline, chlortetracycline, clomocycline, and demeclocycline). Additional examples include cycloserine, mupirocin, tuberin amphomycin, bacitracin, capreomycin, colistin, enduracidin, enviomycin, and 2,4 diaminopyrimidines (e.g., brodimoprim).

Antiviral agents that can be used in combination with an immunogenic composition of the invention include, but are not limited to, non-nucleoside reverse transcriptase inhibitors, nucleoside reverse transcriptase inhibitors, protease inhibitors, and fusion inhibitors. In one embodiment, the antiviral agent is selected from the group consisting of amantadine, oseltamivir phosphate, rimantadine, and zanamivir. In another embodiment, the antiviral agent is a non-nucleoside reverse transcriptase inhibitor selected from the group consisting of delavirdine, efavirenz, and nevirapine. In another embodiment, the antiviral agent is a nucleoside reverse transcriptase inhibitor selected from the group consisting of abacavir, didanosine, emtricitabine, emtricitabine, lamivudine, stavudine, tenofovir DF, zalcitabine, and zidovudine. In another embodiment, the antiviral agent is a protease inhibitor selected from the group consisting of amprenavir, atazanavir, fosamprenav, indinavir, lopinavir, nelfinavir, ritonavir, and saquinavir. In another embodiment, the antiviral agent is a fusion inhibitor such as enfuvirtide. Additional, non-limiting examples of antiviral agents for use in combination with an immunogenic composition of the invention include the following: rifampicin, nucleoside reverse transcriptase inhibitors (e.g., AZT, ddT, ddC, 3TC, d4T), non-nucleoside reverse transcriptase inhibitors (e.g., delavirdine efavirenz, nevirapine), protease inhibitors (e.g., aprenavir, indinavir, ritonavir, and saquinavir), idoxuridine, cidofovir, acyclovir, ganciclovir, zanamivir, amantadine, and palivizumab. Other examples of anti-viral agents include but are not limited to acemannan; acyclovir; acyclovir sodium; adefovir; alovudine; alvircept sudotox; amantadine hydrochloride (SYMMETREL™); aranotin; arildone; atevirdine mesylate; pyridine; cidofovir; cipamfylline; cytarabine hydrochloride; delavirdine mesylate; desciclovir; didanosine; disoxaril; edoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; oseltamivir phosphate (TAMIFLU™); penciclovir; pirodavir; ribavirin; rimantadine hydrochloride (FLUMADINE™); saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zanamivir (RELENZA™); zidovudine; and zinviroxime.

5.5.3 Methods of Administering the Conjugates or Immunogenic Compositions of the Invention

Immunogenic compositions of the invention can be administered to a patient, preferably a mammal, more preferably a human, suffering from a disease or condition that may be amendable to immunotherapy. In a specific embodiment, an immunogenic composition of the invention comprising a conjugate as described herein or a pharmaceutically acceptable salt thereof, is administered to a patient, preferably a mammal, more preferably a human, as a preventative measure against a disease or condition, e.g., a drug addiction. In another embodiment, an immunogenic composition of the invention comprising a conjugate as described herein or a pharmaceutically acceptable salt thereof, is administered to a patient, preferably a human, to prevent disease in a subject which has not yet had the disease or condition.

Immunogenic compositions of the invention can be administered to a subject, preferably a mammal, more preferably a human, suffering from a disease or condition to be targeted by the immunogenic composition. In a specific embodiment, an immunogenic composition of the invention comprising a conjugate described herein or a pharmaceutically acceptable salt thereof, is administered to a subject, preferably a mammal, more preferably a human, as a preventative measure against such a disease or condition.

When administered to a patient, an immunogenic composition of the invention comprising a conjugate described herein or a pharmaceutically acceptable salt thereof is preferably administered as component of a composition that optionally comprises a pharmaceutically acceptable vehicle. The composition can be administered orally, nasally, by inhalation, or by any other convenient route, for example, by infusion or bolus injection, by absorption through epithelial or mucocutaneous linings (e.g., oral mucosa, rectal, and intestinal mucosa) and may be administered together with another biologically active agent. Administration can be systemic or local. Various delivery systems are known, e.g., as an aerosol or by encapsulation in liposomes, microparticles, microcapsules, capsules, and can be used to administer an immunogenic composition of the invention comprising a conjugate described herein or pharmaceutically acceptable salts thereof.

Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a compound of the invention or a pharmaceutically acceptable salt thereof into the bloodstream.

In specific embodiments, it may be desirable to administer an immunogenic composition of the invention comprising a conjugate described herein or a pharmaceutically acceptable salt thereof locally. This may be achieved, for example, and not by way of limitation, by local infusion, topical application, e.g., in conjunction with a wound dressing, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce an immunogenic composition of the invention comprising a conjugate described herein or a pharmaceutically acceptable salt thereof into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, an immunogenic composition of the invention comprising a conjugate described herein or a pharmaceutically acceptable salt thereof is formulated as a suppository, with traditional binders and vehicles such as triglycerides.

In another embodiment, an immunogenic composition of the invention comprising a conjugate described herein or a pharmaceutically acceptable salt thereof is delivered in a vesicle, in particular a liposome (see Langer, 1990, Science 249:1527 1533; Treat et al., in Liposomes in the Therapy of Infectious Disease and Bacterial infection, Lopez-Berestein and Fidler (eds.), Liss, New York, pp. 353 365 (1989); Lopez Berestein, ibid., pp. 317 327; see generally ibid.).

In another embodiment, an immunogenic composition of the invention comprising a conjugate described herein or a pharmaceutically acceptable salt thereof is delivered in a controlled release system (see, e.g., Goodson, in Medical Applications of Controlled Release, supra, vol. 2, pp. 115 138 (1984)). Examples of controlled-release systems are discussed in the review by Langer, 1990, Science 249:1527 1533 may be used. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In a specific embodiment, a controlled-release system comprising an immunogenic composition of the invention comprising a conjugate described herein or a pharmaceutically acceptable salt thereof is placed in close proximity to the viral infection to be prevented, treated and/or managed. In accordance with this embodiment, the close proximity of the controlled-release system to the infection may result in only a fraction of the dose of the composition required if it is systemically administered.

5.5.4 Dosages and Frequency

The amount of a conjugate of the invention, or the amount of an immunogenic composition comprising the conjugate, that will be effective in the prevention, treatment and/or management of a disease or condition can be determined by standard clinical techniques. In vitro or in vivo assays may optionally be employed to help identify optimal dosage ranges. The precise dose to be employed will also depend, e.g., on the route of administration, the type of disease or condition to be treated, and the seriousness of the disease or condition, and should be decided according to the judgment of the practitioner and each patient's or subject's circumstances.

Exemplary doses of the conjugates, antibodies raised in response to the conjugates, or immunogenic compositions of the invention include milligram or microgram amounts per kilogram of subject or sample weight (e.g., about 1 microgram per kilogram to about 500 milligrams per kilogram, about 5 micrograms per kilogram to about 100 milligrams per kilogram, or about 1 microgram per kilogram to about 50 micrograms per kilogram). In specific embodiments, a daily dose is at least 50 mg, 75 mg, 100 mg, 150 mg, 250 mg, 500 mg, 750 mg, or at least 1 g.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM.

In one embodiment, the dosage is a concentration of 0.01 to 5000 mM, 1 to 300 mM, 10 to 100 mM and 10 mM to 1 M. In another embodiment, the dosage is a concentration of at least 5 μM, at least 10 μM, at least 50 μM, at least 100 μM, at least 500 μM, at least 1 mM, at least 5 mM, at least 10 mM, at least 50 mM, at least 100 mM, or at least 500 mM. In a specific embodiment, the dosage is 0.25 μg/kg or more, preferably 0.5 μg/kg or more, 1 μg/kg or more, 2 μg/kg or more, 3 μg/kg or more, 4 μg/kg or more, 5 μg/kg or more, 6 μg/kg or more, 7 μg/kg or more, 8 μg/kg or more, 9 μg/kg or more, or 10 μg/kg or more, 25 μg/kg or more, preferably 50 μg/kg or more, 100 μg/kg or more, 250 μg/kg or more, 500 μg/kg or more, 1 mg/kg or more, 5 mg/kg or more, 6 mg/kg or more, 7 mg/kg or more, 8 mg/kg or more, 9 mg/kg or more, or 10 mg/kg or more of a patient's body weight.

In another embodiment, the dosage is a unit dose of 10-20 μg, 20-50 μg, 50-75 μg, 75-100 μg, 100-200 μg, 200-300 μg, 300-400 μg, 400-500 μg, 500-600 μg, 600-700 μg, 700-800 μg, 800-900 μg, or 900-1000 μg, or more. In some embodiments, the dosage is a unit dose of 5 mg, preferably 10 mg, 50 mg, 100 mg, 150 mg, 200 mg, 250 mg, 300 mg, 350 mg, 400 mg, 500 mg, 550 mg, 600 mg, 650 mg, 700 mg, 750 mg, 800 mg or more. In another embodiment, the dosage is a unit dose that ranges from about 5 mg to about 100 mg, preferably about 100 mg to about 200 μg, about 150 mg to about 300 mg, about 150 mg to about 400 mg, 250 μg to about 500 mg, about 500 mg to about 800 mg, about 500 mg to about 1000 mg, or about 5 mg to about 1000 mg.

In certain embodiments, suitable dosage ranges for oral administration are about 0.001 milligram to about 500 milligrams of a conjugate, antibody, or immunogenic composition of the invention, or a pharmaceutically acceptable salt thereof, per kilogram body weight per day. In specific embodiments of the invention, the oral dose is about 0.01 milligram to about 100 milligrams per kilogram body weight per day, about 0.1 milligram to about 75 milligrams per kilogram body weight per day or about 0.5 milligram to 5 milligrams per kilogram body weight per day. The dosage amounts described herein refer to total amounts administered; that is, if more than one compound is administered, then, in some embodiments, the dosages correspond to the total amount administered. In a specific embodiment, oral compositions contain about 10% to about 95% a compound of the invention by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. In some embodiments, suitable dosage ranges for intranasal administration are about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 500 milligrams per kilogram of body weight per day. Suitable doses for topical administration include doses that are in the range of about 0.001 milligram to about 50 milligrams, depending on the area of administration. Effective doses may be extrapolated from dose-response curves derived from in vitro or animal model test systems. Such animal models and systems are well known in the art.

In another embodiment, a subject is administered one or more doses of a prophylactically or therapeutically effective amount of a conjugate of the invention, an antibody raised in response to the conjugate, or an immunogenic composition of the invention, wherein the prophylactically or therapeutically effective amount is not the same for each dose. In another embodiment, a subject is administered one or more doses of a prophylactically or therapeutically effective amount of conjugate of the invention, an antibody raised in response to the conjugate, or an immunogenic composition of the invention, wherein the dose of a prophylactically or therapeutically effective amount administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses. In another embodiment, a subject is administered one or more doses of a prophylactically or therapeutically effective amount of a conjugate of the invention, an antibody raised in response to the conjugate, or an immunogenic composition of the invention, wherein the dose is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, or 50 μg/kg, as treatment progresses.

In certain embodiments, a subject is administered one or more doses of an effective amount of a conjugate of the invention, an antibody raised in response to the conjugate, or an immunogenic composition of the invention, wherein the dose of an effective amount inhibits or reduces the level or hapten in the body or circulating antigen by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85%. In other embodiments, a subject is administered one or more doses of an effective amount of a conjugate of the invention, an antibody raised in response to the conjugate, or an immunogenic composition of the invention, wherein the dose of an effective amount inhibits or reduces the severity of the disease or condition, e.g., a drug addiction, by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85%.

In other embodiments, a subject is administered one or more doses of an effective amount of a conjugate of the invention, an antibody raised in response to the conjugate, or an immunogenic composition of the invention, wherein the dose of an effective amount inhibits or reduces nicotine or cocaine addiction by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85%. In other embodiments, a subject is administered one or more doses of an effective amount of a conjugate of the invention, an antibody raised in response to the conjugate, or an immunogenic composition of the invention, wherein the dose of an effective amount inhibits or reduces the ability of the antigen targeted by the conjugate to other cells, tissues or organs in the subject by at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85%.

The dosages of prophylactic or therapeutic agents other than a conjugate of the invention, an antibody raised in response to the conjugate, or an immunogenic composition of the invention which have been or are currently being used for the prevention, treatment and/or management of the disease or condition or a disease or condition related to it can be determined using references available to a clinician such as, e.g., the Physicians' Desk Reference (55th ed. 2001). Preferably, dosages lower than those which have been or are currently being used to prevent, treat and/or manage the disease or condition are utilized in combination with one or more conjugates of the invention, antibodies raised in response to the conjugate, or immunogenic compositions of the invention.

The above-described administration schedules are provided for illustrative purposes only and should not be considered limiting. A person of ordinary skill in the art will readily understand that all doses are within the scope of the invention.

5.5.5 Kits

The invention provides a pharmaceutical pack or kit comprising one or more containers filled with a conjugate of the invention, an antibody raised in response to the conjugate, or an immunogenic composition of the invention. The kits can be used in the above-described methods. In particular, the kits can be used for the prevention, treatment, and/or management of a disease or condition, e.g., a drug addiction (e.g., to cocaine or nicotine).

In one embodiment, a kit comprises a conjugate of the invention, an antibody raised in response to the conjugate, or an immunogenic composition of the invention, in one or more containers. In another embodiment, a kit comprises a conjugate of the invention, an antibody raised in response to the conjugate, or an immunogenic composition of the invention, in one or more containers, and one or more other prophylactic or therapeutic agents, in one or more other containers. In a particular embodiment, the kit further comprises instructions for using the conjugate of the invention, antibody raised in response to the conjugate, or immunogenic composition of the invention, as well as an explanation of side effects of the conjugate of the invention, antibody raised in response to the conjugate, or immunogenic composition of the invention, and dosage information for a particular route of administration. Optionally associated with such container(s) can be a notice in the form prescribed by a governmental agency regulating the manufacture, use or sale of pharmaceuticals or biological products, which notice reflects approval by the agency of manufacture, use or sale for human administration.

5.5.6 Immunogenic Compositions for Inducing an Immune Response to a Drug of Addiction

The immunogenic compositions of the invention are exemplified in this section using examples from experiments with drug (cocaine or nicotine) hapten-carrier conjugates. These teachings can be adapted for use with antigen-carrier and hapten-carrier conjugates comprising any antigen or hapten, respectively, to raise an immune response against any of the antigens or a hapten described supra, or any other antigen for which an immune response, particularly an improved immune response, is desired.

The drug-conjugates of the present invention, as well as the compositions of the present invention, may also be used as a therapeutic, to treat a drug addiction or drug dependence, or as a prophylactic. In prophylactic use, the drug-conjugates or immunogenic compositions comprising them may be administered to a mammal prior to any exposure to the drug to generate anti-drug antibodies. The generated anti-drug antibodies would be present in the mammal to bind to any drug introduced subsequent to the administration of the immunogenic composition, and therefore minimize or prevent the chance of becoming addicted to the drug.

The immunogenic compositions of the instant invention may be used in a subject as vaccines. These compositions containing at least one drug/hapten-carrier conjugate capable of eliciting the production of a sufficiently high titer of antibodies specific to the drug/hapten such that upon subsequent challenge with the drug/hapten said antibodies are capable of reducing the addictive properties of the drug. The expected immune response to a hapten-carrier conjugate is the formation of both anti-hapten and anti-carrier antibodies. The therapeutic level is reached when a sufficient amount of the anti-drug specific antibodies are elicited and maintained to mount a neutralizing attack on drug introduced after vaccination. The therapeutic regimens of the instant invention allow for sufficient time for production of antibodies after initial vaccination and any boosting. Further, the optimal anti-drug vaccine contains at least one drug/hapten carrier conjugate comprising an optimal combination of the drug as hapten and a carrier so that production of anti-drug antibodies is capable of achieving an optimal therapeutic level, that is, remaining in vivo at a sufficiently high titer to withstand a subsequent challenge within several months with the selected drug. More particularly, the antibody titers remain sufficiently high to provide an effective response upon subsequent exposure to the drug for about two months to about one year or more depending upon the individual, more usually at least three months. This optimal composition consists of a hapten-carrier conjugate, excipients and, optionally adjuvants.

When used in the treatment of nicotine, the present invention defines a hapten-carrier conjugate, wherein the hapten is nicotine or a nicotine derivative, which can be used to immunize mammals, particularly humans, to elicit anti-nicotine antibodies capable of binding free drug and preventing transit of the drug to the reward system in the brain thereby abrogating addictive drug-taking behavior (e.g., smoking cigarettes). It is believed that nicotine binds to the alpha-subunit of the nicotinic acetylcholine receptors in the brain which results in an increase in dopamine release. It is thought that increased numbers of nicotinic acetylcholine receptors in the brain enhance the physiological dependence of nicotine. As discussed above in relation to cocaine, anti-nicotine antibodies would presumably limit the distribution of nicotine across the blood-brain barrier to the brain, thus reducing its pharmacological effects. Antibody intervention in the case of nicotine, however, may have some advantages over cocaine. For example, there is some level of standardization with nicotine delivery; that is, each cigarette contains on average 9 mg of nicotine of which 1-3 mg are effectively dispensed during smoking Additionally, the peak plasma concentration of nicotine is 25-50 ng/ml which is significantly lower than that of cocaine (0.3-1 μg/ml). This should provide an ideal opportunity for intervention with moderately high affinity antibodies.

Initial vaccination with the immunogenic hapten-carrier conjugate composition of the present invention creates high titers of hapten-specific antibodies in vivo. Periodic tests of the vaccinated subjects plasma are useful to determine individual effective doses. Titer levels are increased and maintained through periodic boosting. It is anticipated that this therapeutic will be used in combination with current drug rehabilitation programs, including counseling. Further, the therapeutic compositions of the present invention may be aimed at a single drug or several drugs simultaneously or in succession and may be used in combination with other therapies. For example, the therapeutic hapten-carrier conjugate compositions and methods of the instant invention are used without adverse interactions in combination with conventional pharmacological approaches and previously discussed “short term” passive immunization to enhance the overall effect of therapy.

The immunogenic hapten-carrier conjugate composition of the present invention is prepared by coupling one or more hapten molecules to a T cell epitope containing carrier to obtain a hapten-carrier conjugate capable of stimulating T cells (immunogenic) which leads to T cell proliferation and a characteristic release of mediators which activate relevant B cells and stimulate specific antibody production.

Antibodies of interest are those specific to the hapten portion of the hapten-carrier conjugate (also called the hapten-carrier complex). Therapeutic compositions containing a combination of conjugates, either to the same drug (cross-immunization) or to multiple drugs (co-immunization) are disclosed. Such co-mixtures of conjugates of multiple drugs are particularly useful in the treatment of polydrug abuse.

In selecting a drug suitable for conjugation according to the instant invention, one skilled in the art would select drug with properties likely to elicit high antibody titers. However, if the chosen molecule is similar to those molecules which are endogenous to the individual, antibodies raised against such a molecule could cross-react with many different molecules in the body giving an undesired effect. Thus, the drug to be selected as the hapten (drug/hapten) must be sufficiently foreign and of a sufficient size so as to avoid eliciting antibodies to molecules commonly found inside a human body. For these reasons, alcohol, for example, would not be suitable for the therapeutic of the instant invention. The antibodies raised against the therapeutic composition are highly specific and of a sufficient quantity to neutralize the drug either in the blood stream or in the mucosa or both. Without limiting the invention, the drugs which are suitable for therapeutic composition (not in order of importance) are:

Hallucinogens, for example mescaline and LSD;

Cannabinoids, for example THC;

Stimulants, for example amphetamines, cocaine, phenmetrazine, methylphenidate;

Nicotine;

Depressants, for example, nonbarbiturates (e.g. bromides, chloral hydrate etc.), methaqualone, barbiturates, diazepam, flurazepam, phencyclidine, and fluoxetine;

Opium and its derivatives, for example, heroin, methadone, morphine, meperidine, codeine, pentazocine, and propoxyphene; and

“Designer drugs” such as “ecstasy”.

5.5.6.1 Use with Adjuvants

For the purposes of the present invention, an adjuvant is used to enhance the immune response to a specific antigen or a hapten, e.g., when an adjuvant is co-administered with an immunogenic composition, the immune response is greater than the immune response elicited by an equivalent amount of the immunogenic composition administered without the adjuvant, or the adjuvant is used to direct a particular type or class of immune response against a co-administered antigen. Co-administration of an “effective amount” of an adjuvant of the present invention will be that amount which enhances an immunological response to the co-administered immunogenic composition such that, for example, lower or fewer doses of the immunogenic composition are required to generate an efficient immune response.

As used herein, the term “co-administered” intends either the simultaneous or concurrent administration of the adjuvant and the immunogenic composition, e.g., when the two are present in the same composition or administered in separate compositions at nearly the same time but at different sites, as well as the delivery of the adjuvant and immunogenic composition in separate compositions at different times, including delivery to different sites. For example, the adjuvant may be delivered prior or subsequent to delivery of the immunogenic composition at the same or a different site. The timing between adjuvant and immunogenic composition deliveries can range from about several minutes apart, to several hours apart, to several days apart.

Any adjuvant which does not mask the effect of the carrier is considered useful in the immunogenic compositions of the present invention (see, Edelman (1980) Rev. Infect. Dis. 2:370-373). Initial experiments aimed at demonstrating the feasibility of a therapeutic vaccine against cocaine addiction used the powerful adjuvant CFA. However, CFA is not preferred in humans. A useful adjuvant currently licensed for use in humans is alum, including aluminum hydroxide (Spectrum Chem. Mtg. Corp., New Brunswick, N.J.) or aluminum phosphate (Spectrum). Typically, the vaccine is adsorbed onto the alum, which has very limited solubility. Preliminary data in a murine model suggest that alum is capable of inducing a strong anti-cocaine antibody response, and MF59 (Chiron, Emeryville, Calif.) or RIBI adjuvant is also suitable.

It is notable that effective immunization with CTB as the carrier protein does not require a powerful adjuvant. High titer anti-cocaine antibody responses were induced by immunization with the CTB-cocaine conjugate either using alum as the adjuvant or in the absence of any added adjuvant. For carriers other than CTB one skilled in the art would be capable of determining an appropriate adjuvant, if needed.

The use of adjuvant is often beneficial in immunization protocols. To assess the contribution of alum to the immune response, mice were immunized with 10 μg cocaine-CTB PS-5.53 intraperitoneally in saline or adsorbed onto alum. The mice were boosted at day 27 using the same protocol. For both groups of animals, high levels of cocaine-specific antibodies were detected by day 43 (titer of 14687 without alum and 16775 with alum). Immunization with cocaine-CTB adsorbed onto alum has also been shown to be effective with a subcutaneous or intramuscular route of administration. Therefore, the use of alum is acceptable with this antigen.

The addition of alum adjuvant can increase the immune response to injected proteins obtaining sufficient antibody titers requires testing the contribution of alum to the antibody response after injection of drug-carrier conjugates. To assess the contribution of alum mice were immunized with 10 μg cocaine-rCTB PS-5.189, where the CTB was recombinantly expressed in bacteria. The mice were injected intramuscularly in saline or absorbed onto alum and again on day 14. For these lots of cocaine-CTB conjugates the addition of alum is required in order to generate anti-cocaine antibodies as detected by ELISA.

5.5.6.2 Excipients and Auxiliary Agents

The immunogenic compositions of the invention may optionally contain one or more pharmaceutically acceptable excipients including, but not limited to, sterile water, salt solutions such as saline, sodium phosphate, sodium chloride, alcohol, gum arabic, vegetable oils, benzyl alcohols, polyethylene glycol, gelatine, mannitol, carbohydrates, magnesium stearate, viscous paraffin, fatty acid esters, hydroxy methyl cellulose and buffer. Other suitable excipients may be used by those skilled in that art. The therapeutic composition may optionally comprising at least one auxiliary agent, for example, dispersion media, coatings, such as lipids and liposomes, surfactants such as wetting agents and emulsifiers, lubricants, preservatives such as antibacterial agents and anti fungal agents, stabilizers and other agents well known to those skilled in the art. The compositions of the present invention may also contain further adjuvants, agents and/or inert pharmacologically acceptable excipients which may be added to enhance the therapeutic properties of the drug or enable alternative modes of administration.

Highly purified hapten-carrier conjugates produced as discussed above may be formulated into immunogenic compositions of the invention suitable for human therapy. If a therapeutic composition of the invention is to be administered by injection (i.e., subcutaneous injection), then it is preferable that the highly purified hapten-carrier conjugate be soluble in aqueous solution at a pharmaceutically acceptable pH (that is, a range of about 4-9) such that the composition is fluid and easy administration exists. It is possible, however, to administer a composition wherein the highly purified hapten-carrier conjugate is in suspension in aqueous solution and such a suspension is within the scope of the present invention. The composition also optionally includes pharmaceutically acceptable excipients, adjuvant and auxiliary agents or supplementary active compounds. Depending upon the mode of administration, optional ingredients would ensure desirable properties of the therapeutic composition, for example, proper fluidity, prevention of action of undesirable microorganisms, enhanced bioavailability or prolonged absorption.

An immunogenic composition of the invention should be sterile, stable under conditions of manufacture, storage, distribution and use, and preserved against the contaminating action of microorganisms such as bacteria and fungi. A preferred means for manufacturing a therapeutic composition of the invention in order to maintain the integrity of the composition is to prepare the formulation of conjugate and pharmaceutically excipient such that the composition may be in the form of a lyophilized powder which is reconstituted in excipients or auxiliary agents, for example sterile water, just prior to use. In the case of sterile powders for the preparation of sterile injectable solutions, the preferred methods of preparation are vacuum drying, freeze-drying or spin drying which yields a powder of the active ingredient plus any additional desired ingredient from a previously sterile-filtered solution thereof.

The active compounds of this invention can be processed in accordance with conventional methods of galenic pharmacy to produce therapeutic compositions for administration to patients, e.g., mammals including humans. The preferred modes of administration are intranasal, intratracheal, oral, dermal, and/or injection. One particularly suitable combination of modes of administration comprises an initial injection with intranasal boosts.

For parenteral application, particularly suitable are injectable, sterile solutions, preferably oily or aqueous solutions, as well as suspensions, emulsions, or implants, including suppositories. Ampoules are convenient unit dosages. For enteral application, particularly suitable are tablets, dragees, liquids, suspensions, drops, suppositories, or capsules, which may include enteric coating. A syrup, elixir, or the like can be used wherein a sweetened vehicle is employed.

Sustained or directed release compositions can be formulated, e.g., liposomes or those wherein the active compound (conjugate) is protected with differentially degradable coatings, e.g., by microencapsulation, multiple coatings, etc. It is also possible to freeze-dry the new compounds and use the lyophilizates obtained, for example, for the preparation of products for injection.

For topical application, there are employed as nonsprayable forms, viscous to semi-solid or solid forms comprising a carrier compatible with topical application and having a dynamic viscosity preferably greater than water. Suitable formulations include but are not limited to solutions, suspensions, emulsions, creams, ointments etc., which are, if desired, sterilized or mixed with auxiliary agent. For topical application suitable are sprayable aerosol preparations wherein the active compound, preferably in combination with a suitable excipient or auxiliary agent, is packaged in a squeeze bottle or in admixture with a pressurized volatile, normally gaseous propellant.

An antibody raised through the compositions and methods of the instant invention may have a molecular weight ranging from 150 KDa to 1,000 KDa. When the subject is exposed to free cocaine or nicotine after vaccination with the optimized conjugate in the therapeutic composition, the free cocaine or nicotine is targeted by cocaine-specific or nicotine-specific antibody or antibodies. No changes in the form or structure of the drug are necessary for the antibody to recognize the drug in vivo. While not intending to limit the present invention, it is believed that upon exposure of the vaccinated individual to cocaine or nicotine, the anti-drug antibodies will block the effects of cocaine and nicotine. At least three mechanisms are believed to contribute to the blocking activity. First, antibodies are unable to cross the blood-brain barrier. Therefore, it is believed that cocaine or nicotine, when bound to the anti-cocaine or anti-nicotine antibody, will not cross the blood-brain barrier and will not be able to exert its effect on dopamine transporters. Second, the antibody prevents the drug from binding to its receptor by simple steric blockade.

This mechanism is expected to be operative in blocking some of the non-CNS effects of the drugs (e.g. cardiac toxicity) and in the activity of antibodies against other drugs with non-CNS targets. Third, both cocaine and nicotine have relatively short half-lives in vivo due to both enzymatic and non-enzymatic degradation, creating inactive metabolites. Cocaine and nicotine, in particular, are sufficiently small drugs so that it is very unlikely that they could cross-link antibodies, thus, it is highly unlikely that physiologically significant immune complex formation will occur for either of the drugs.

Still further embodiments of mucosal applications are used in the practice of the present invention. For example, copolymer microspheres are used to induce or enhance a mucosal immune response. These small, biodegradable microspheres encapsulate and protect the conjugate and facilitate uptake by the mucosal immune system. Although they are most widely used for oral immunization, they also have been reported to be effective with intranasal immunization (Walker (1994) Vaccine 12:387-399). Inert polymers such as poly(lactide-co-glycolide) (PLG) of 1-10 μm diameter are particularly useful in this regard (Holmgren et al. (1994) Am. J. Trop. Med. Hyg. 50:42-54; Serva (1994) Science 265:1522-1524).

In addition to the preferred conjugates, cross-immunization with different conjugates is carried out in order to minimize antibody cross-reactivity. Mice are primed with conjugates, more particularly bacterial toxin carrier conjugates, and then boosted at day 14 with a reciprocal conjugate coupled to a different carrier, BSA. Only the subset of antibody-secreting B cells that recognize both of the cocaine conjugates are maximally stimulated and expanded. It is believed that because the two conjugates differ in their point of attachment to the cocaine molecule, the specificity of the recognition increases. Specificity of the induced antisera is then confirmed by competition ELISA.

Still further, immunogenic compositions containing more than one conjugate stimulate polyclonal antibodies thereby enhancing antibody response upon subsequent challenge.

### 6. EXAMPLE

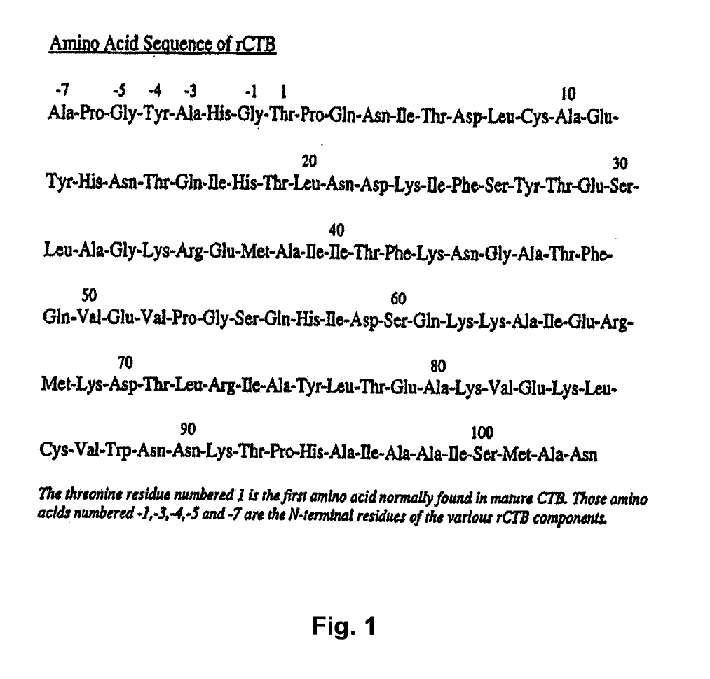

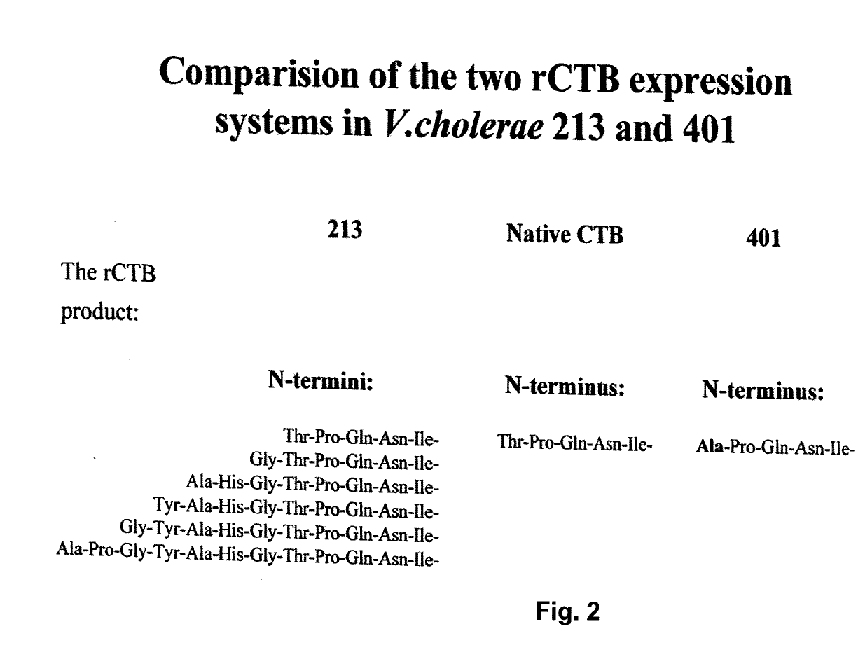

This example demonstrates that immunogenic compositions comprising nicotine as a hapten and rCTB as carrier in a hapten-carrier conjugate have enhanced immunogenicity when the rCTB contains a signal peptide. rCTB is translated with a signal peptide with the amino acid sequence Ala-Pro-Gly-Tyr-Ala-His-Gly (FIG. 1). This signal sequence is not present in mature CTB. rCTB was produced in two expression systems, the V. cholerae strains 213 and 401. When produced in the 213 strain, forms of rCTB are detected that contain a signal sequence of up to 7 amino acids in length. No stable signal sequence is detected when rCTB is expressed in the 401 strain, but rather, the amino-terminus is an alanine residue instead of the threonine normally present in native, mature CTB (FIG. 2). Nicotine-CTB conjugates containing rCTB from the 213 strain have a two-fold enhanced immunogenicity compared to conjugates generated using rCTB from the 401 strain, demonstrating that signal peptide-containing bacterial toxins, when used as carriers in nicotine-carrier conjugates, have enhanced immunogenicity.

### 7. EQUIVALENTS

Those skilled in the art will recognize or be able to ascertain, using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

### 8. ILLUSTRATIVE EMBODIMENTS

The invention can be illustrated by the non-limiting, embodiments set forth in the following paragraphs.
1. An immunogenic composition comprising a bacterial toxin containing a signal peptide and an antigen.
2. An immunogenic composition comprising a bacterial toxin containing a signal peptide and an antigen, wherein the composition is capable of inducing an immune response to an antigen.
3. An immunogenic composition comprising a bacterial toxin containing a signal peptide and a hapten.
4. An immunogenic composition comprising a bacterial toxin containing a signal peptide and a hapten, wherein the composition is capable of inducing an immune response to a drug of addiction.
5. An immunogenic composition comprising a bacterial toxin containing a signal peptide and nicotine or a derivative thereof, wherein the composition is capable of inducing an immune response to nicotine.
6. An immunogenic composition comprising (i) cholera toxin B (CTB) with a signal peptide comprising the amino acid sequence Ala-Pro-Gly-Tyr-Ala-His-Gly, or a C-terminal fragment thereof; and (ii) nicotine or a derivative thereof, wherein the composition is capable of inducing an immune response to nicotine.
7. The immunogenic composition of paragraph 6, wherein the immune response to nicotine is greater than the immune response to nicotine induced by an immunogenic composition identical to that of paragraph 6 but lacking said signal peptide.
8. The immunogenic composition of any one of paragraphs 1-3, wherein the bacterial toxin containing a signal peptide is cholera toxin B (CTB).
9. The immunogenic composition of paragraph 8, wherein the CTB signal peptide comprises the amino acid sequence Ala-Pro-Gly-Tyr-Ala-His-Gly, or a C-terminal fragment thereof
10. The immunogenic composition of paragraph 4, wherein the bacterial toxin containing a signal peptide is cholera toxin B (CTB).
11. The immunogenic composition of paragraph 10, wherein the CTB signal peptide comprises the amino acid sequence Ala-Pro-Gly-Tyr-Ala-His-Gly, or a C-terminal fragment thereof
12. The immunogenic composition of any one of paragraphs 1-5, wherein the bacterial toxin is E. coli heat labile enterotoxin subunit B.
13. The immunogenic composition of any one of paragraphs 1-5, wherein the bacterial toxin is diphtheria toxin.
14. The immunogenic composition of any one of paragraphs 1-5, wherein the bacterial toxin is a pertussis toxin.
15. The immunogenic composition of any one of paragraphs 1-5, wherein the bacterial toxin is a shiga toxin.
16. The immunogenic composition of any one of paragraphs 1-5, wherein the bacterial toxin is Pseudomonas exotoxin A.
17. The immunogenic composition of any one of paragraphs 1-5, wherein said immunogenic composition comprises an adjuvant.
18. The immunogenic composition of paragraph 17, wherein said adjuvant is aluminum hydroxide.
19. The immunogenic composition paragraph 6, wherein said immunogenic composition comprises an adjuvant.
20. The immunogenic composition of paragraph 19, wherein said adjuvant is aluminum hydroxide.
21. The immunogenic composition of any one of paragraphs 8, 10 or 12-16, wherein the bacterial toxin contains its endogenous signal peptide, or a fragment thereof.
22. A method of inducing an immune response in a subject, comprising administering to the subject an effective amount of an immunogenic composition of any one of paragraphs 1-21.
23. A method of inducing an immune response against a drug of addiction in a subject, comprising administering to the subject an effective amount of an immunogenic composition of paragraph 4, 10 or 11.
24. The method of paragraph 23, wherein the drug of addiction is nicotine.
25. The method of paragraph 23, wherein the drug of addiction is cocaine.
26. A method of inducing an immune response against nicotine in a subject, comprising administering to the subject an effective amount of an immunogenic composition of paragraph 5.
27. A method of inducing an immune response against nicotine in a subject, comprising administering to the subject an effective amount of an immunogenic composition of paragraph 6 or 7.
28. A method of treating drug addiction or drug dependence in a subject in need thereof, comprising administering to the subject an effective amount of an immunogenic composition of paragraph 4, 10 or 11.
29. The method of paragraph 28, wherein the drug addiction or dependence is nicotine addiction or dependence.
30. The method of paragraph 28, wherein the drug addiction or dependence is cocaine addiction or dependence.
31. A method of treating nicotine addiction or nicotine dependence in a subject in need thereof, comprising administering to the subject an effective amount of an immunogenic composition of paragraph 5.
32. A method of treating nicotine addiction or nicotine dependence in a subject in need thereof, comprising administering to the subject an effective amount of an immunogenic composition of paragraph 6 or 7.
33. A method of vaccinating a subject against a drug of addiction, comprising administering to the subject an effective amount of an immunogenic composition of paragraph 4, 10 or 11.
34. The method of paragraph 33, wherein the drug of addiction is nicotine addiction.
35. The method of paragraph 33, wherein the drug of addiction is cocaine.
36. A method of vaccinating a subject against nicotine, comprising administering to the subject an effective amount of an immunogenic composition of paragraph 5.
37. A method of vaccinating a subject against nicotine, comprising administering to the subject an effective amount of an immunogenic composition of paragraphs 6 or 7.

1. An immunogenic composition comprising (i) cholera toxin B (CTB) with a signal peptide comprising the amino acid sequence Ala-Pro-Gly-Tyr-Ala-His-Gly, or a C-terminal fragment thereof; and (ii) a hapten, wherein the composition is capable of inducing an immune response to a drug of addiction.

2. The immunogenic composition of claim 1, wherein the immune response to the drug of addiction is greater than the immune response to the drug of addition induced by an immunogenic composition identical to that of claim 1 but lacking said signal peptide.

3. The immunogenic composition of claim 1, wherein the drug of addiction is cocaine.

4. (canceled)

5. The immunogenic composition of claim 3, wherein said immunogenic composition comprises an adjuvant.

6. The immunogenic composition of claim 5, wherein said adjuvant is aluminum hydroxide.

7. (canceled)

8. (canceled)

9. (canceled)

10. (canceled)

11. A method of inducing an immune response against a drug of addiction in a subject, comprising administering to the subject an effective amount of an immunogenic composition comprising (i) cholera toxin B (CTB) with a signal peptide comprising the amino acid sequence Ala-Pro-Gly-Tyr-Ala-His-Gly, or a C-terminal fragment thereof; and (ii) a hapten, wherein the composition is capable of inducing an immune response to a drug of addiction.

12. (canceled)

13. The method of claim 11, wherein the drug of addiction is cocaine.

14. (canceled)

15. The method of claim 13, wherein the immunogenic composition is administered with an adjuvant.

16. The method of claim 15, wherein said adjuvant is aluminum hydroxide.

17. A method of vaccinating a subject against a drug of addiction, comprising administering to the subject an effective amount of an immunogenic composition comprising (i) cholera toxin B (CTB) with a signal peptide comprising the amino acid sequence Ala-Pro-Gly-Tyr-Ala-His-Gly, or a C-terminal fragment thereof; and (ii) a hapten, wherein the composition is capable of inducing an immune response to a drug of addiction.

18. (canceled)

19. The method of claim 17, wherein the drug of addiction is cocaine.

20. (canceled)

21. The method of claim 19, wherein the immunogenic composition is administered with an adjuvant.

22. The method of claim 21, wherein said adjuvant is aluminum hydroxide.

23. A method of treating drug addiction or drug dependence in a subject in need thereof, comprising administering to the subject an effective amount of an immunogenic composition comprising (i) cholera toxin B (CTB) with a signal peptide comprising the amino acid sequence Ala-Pro-Gly-Tyr-Ala-His-Gly, or a C-terminal fragment thereof; and (ii) a hapten, wherein the composition is capable of inducing an immune response to a drug of addiction.

24. (canceled)

25. The method of claim 23, wherein the drug addiction or dependence is cocaine addiction or dependence.

26. (canceled)

27. The method of claim 25, wherein the immunogenic composition is administered with an adjuvant, wherein said adjuvant is aluminum hydroxide.

28. The method of claim 27, wherein said adjuvant is aluminum hydroxide.

29. The composition of claim 2, wherein the drug of addiction is cocaine.

30. The method of claim 11, wherein the immune response to the drug of addiction is greater than the immune response to the drug of addition induced by an immunogenic composition identical to that of claim 11 but lacking said signal peptide.

31. The method of claim 23, wherein the immune response to the drug of addiction is greater than the immune response to the drug of addition induced by an immunogenic composition identical to that of claim 23 but lacking said signal peptide.
